SEQUENCE LISTING (86 sequences)

SEQ ID NO: 1 - DNA, 573 bp, Homo sapiens
ttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttc

SEQ ID NO: 2 - protein, 191 aa, Homo sapiens [sequence omitted]

SEQ ID NO: 3 - DNA, 38 bp, Artificial
  synthetic sequence
ggaattccatatgttcccaaccattcccttatccaggc

SEQ ID NO: 4 - DNA, 36 bp, Artificial
  synthetic sequence
cgcggatccctagaagccacagctgccctccacaga

SEQ ID NO: 5 - DNA, 19 bp, Artificial
  synthetic sequence
taatacgactcactatagg

SEQ ID NO: 6 - DNA, 21 bp, Artificial
  synthetic sequence
tgctagttattgctcagcggt

SEQ ID NO: 7 - DNA, 588 bp, Artificial
  synthetic sequence
catatgttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctagggatcc

SEQ ID NO: 8 - protein, 192 aa, Artificial
  synthetic sequence
MFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 9 - DNA, 2907 bp, Artificial
  synthetic sequence
ccggatatagttcctcctttcagcaaaaaacccctcaagacccgtttagaggccccaaggggttatgctagttattgctcagcggtggcagcagccaactcagcttcctttcgggctttgtttagcagcctaggtattaatcaattagtggtggtggtggtggtggtggtgctcgagtgcggccgcaagcttgtcgacggagctcgcctgcaggcgcgccaaggcctgtacagaattcggatccccgatatcccatgggactcttgtcgtcgtcatcaccggagccaccaccggtacccagatctgggctgtccatgtgctggcgttcgaatttagcagcagcggtttctttcataccaattgcagtactaccgcgtggcaccagacccgcggagtgatggtgatggtgatgaccagaaccactagtacacacatatgtatatctccttcttaaagttaaacaaaattatttctagaggggaattgttatccgctcacaattcccctatagtgagtcgtattaatttcgcgggatcgagatcgatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcatggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 10 - DNA, 73 bp, Artificial
  synthetic sequence
cgccatatgaaaaagacagctatcgcgattgcagtggcactggctggtttcgctaccgtagcgcaagcttgag

SEQ ID NO: 11 - protein, 21 aa, Escherichia coli
  SIGNAL (1)..(21)
MKKTAIAIAVALAGFATVAQA

SEQ ID NO: 12 - DNA, 42 bp, Artificial
  synthetic sequence
ggacatatgctgaagctttcccaaccattcccttatccaggc

SEQ ID NO: 13 - DNA, 28 bp, Artificial
  synthetic sequence
cgcggatccctccacagagcggcactgc

SEQ ID NO: 14 - DNA, 578 bp, Artificial
  synthetic sequence
catatgctgaagctttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggatcc

SEQ ID NO: 15 - DNA, 630 bp, Artificial
  synthetic sequence
catatgaaaaagacagctatcgcgattgcagtggcactggctggtttcgctaccgtagcgcaagctttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggatcc

SEQ ID NO: 16 - protein, 208 aa, Artificial
  synthetic sequence
MKKTAIAIAVALAGFATVAQAFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEG

SEQ ID NO: 17 - DNA, 570 bp, Artificial
  synthetic sequence
catatgttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggatcc

SEQ ID NO: 18 - protein, 188 aa, Artificial
  synthetic sequence
MFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEG

SEQ ID NO: 19 - DNA, 52 bp, Artificial
  synthetic sequence
taccatatgacatgatcatgtggcttcttcccaaccattcccttatccaggc

SEQ ID NO: 20 - DNA, 588 bp, Artificial
  synthetic sequence
catatgacatgatcatgtggcttcttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggatcc

SEQ ID NO: 21 - protein, 191 aa, Artificial
  synthetic sequence
SCGFFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEG

SEQ ID NO: 22 - DNA, 42 bp, Artificial
  synthetic sequence
tacgaattccattgatcatgtggcttctagtaggtcgacgat

SEQ ID NO: 23 - protein, 4 aa, Artificial
  synthetic sequence
SCGF

SEQ ID NO: 24 - DNA, 51 bp, Artificial
  synthetic sequence
tacgaattccattgatcatgtggcttcaaaaagaaatagtaggtcgacgat

SEQ ID NO: 25 - protein, 7 aa, Artificial
  synthetic sequence
SCGFKKK

SEQ ID NO: 26 - DNA, 1161 bp, Artificial
  synthetic sequence
catatgacatgatcatgtggcttcttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggatcatgtggcttcttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggatcc

SEQ ID NO: 27 - protein, 382 aa, Artificial
  synthetic sequence
SCGFFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEG

SEQ ID NO: 28 - DNA, 1152 bp, Artificial
  synthetic sequence
tgatcatgtggcttcttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggatcatgtggcttcttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggatcc

SEQ ID NO: 29 - protein, 382 aa, Artificial; identical to SEQ ID NO: 27
  synthetic sequence

SEQ ID NO: 30 - DNA, 606 bp, Artificial
  synthetic sequence
catatgttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggatccgaattccattgatcatgtggcttctagtaggtcgac

SEQ ID NO: 31 - DNA, 1737 bp, Artificial
  synthetic sequence
catatgttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggatcatgtggcttcttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggatcatgtggcttcttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggatcatgtggcttctagtaggtcgac

SEQ ID NO: 32 - protein, 574 aa, Artificial
  synthetic sequence
MFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 33 - DNA, 55 bp, Artificial
  synthetic sequence
taccatatgacatgatcatgtggcttcggtttcccaaccattcccttatccaggc

SEQ ID NO: 34 - DNA, 591 bp, Artificial
  synthetic sequence
catatgacatgatcatgtggcttcggtttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggatcc

SEQ ID NO: 35 - protein, 192 aa, Artificial
  synthetic sequence
SCGFGFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEG

SEQ ID NO: 36 - DNA, 1158 bp, Artificial
  synthetic sequence
tgatcatgtggcttcggtttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggatcatgtggcttcggtttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggatcc

SEQ ID NO: 37 - protein, 384 aa, Artificial
  synthetic sequence
SCGFGFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFGFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEG

SEQ ID NO: 38 - DNA, 1743 bp, Artificial
  synthetic sequence
catatgttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggatcatgtggcttcggtttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggatcatgtggcttcggtttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggatcatgtggcttctagtaggtcgac

SEQ ID NO: 39 - protein, 576 aa, Artificial
  synthetic sequence
MFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFGFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFGFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 40 - DNA, 39 bp, Artificial
  synthetic sequence
cgcggatcctcatgagaagccacagctgccctccacaga

SEQ ID NO: 41 - DNA, 591 bp, Artificial
  synthetic sequence
catatgttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctcatgaggatcc

SEQ ID NO: 42 - protein, 193 aa, Artificial [sequence omitted]

SEQ ID NO: 43 - DNA, 50 bp, Artificial
  synthetic sequence
catgccatggggtggtggaggaagtttcccaaccattcccttatccaggc

SEQ ID NO: 44 - DNA, 606 bp, Artificial
  synthetic sequence
ccatggggtggtggaggaagtttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctcatgaggatcc

SEQ ID NO: 45 - protein, 198 aa, Artificial
  synthetic sequence
WGGGGSFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFS

SEQ ID NO: 46 - DNA, 603 bp, Artificial
  synthetic sequence
ccatggggtggtggaggaagtttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctagggatcc

SEQ ID NO: 47 - protein, 197 aa, Artificial
  synthetic sequence
WGGGGSFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 48 - DNA, 1200 bp, Artificial
  synthetic sequence
ccatggggtggtggaggaagtttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctcatggggtggtggaggaagtttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctcatgaggatcc

SEQ ID NO: 49 - protein, 396 aa, Artificial
  synthetic sequence
WGGGGSFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFSWGGGGSFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFS

SEQ ID NO: 50 - DNA, 1185 bp, Artificial
  synthetic sequence
catatgttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctcatggggtggtggaggaagtttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctcatgaggatcc

SEQ ID NO: 51 - protein, 391 aa, Artificial
  synthetic sequence
MFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFSWGGGGSFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFS

SEQ ID NO: 52 - DNA, 1779 bp, Artificial
  synthetic sequence
catatgttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctcatggggtggtggaggaagtttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctcatggggtggtggaggaagtttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctcatgaggatcc

SEQ ID NO: 53 - protein, 589 aa, Artificial
  synthetic sequence
MFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFSWGGGGSFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFSWGGGGSFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFS

SEQ ID NO: 54 - DNA, 2370 bp, Artificial
  synthetic sequence
catatgttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctcatggggtggtggaggaagtttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctcatggggtggtggaggaagtttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctcatggggtggtggaggaagtttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctagggatcc

SEQ ID NO: 55 - protein, 786 aa, Artificial
  synthetic sequence
MFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFSWGGGGSFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFSWGGGGSFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFSWGGGGSFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 56 - DNA, 33 bp, Artificial
  synthetic sequence
ttaccatggattgccggcggcggcggatccaat

SEQ ID NO: 57 - DNA, 36 bp, Artificial
  synthetic sequence
ttaccatggatttgatcaggcggcggcggatccaat

SEQ ID NO: 58 - DNA, 36 bp, Artificial
  synthetic sequence
tgatcaggcggcggcggatcaggcggcggcggatcc

SEQ ID NO: 59 - protein, 10 aa, Artificial
  synthetic sequence
SGGGGSGGGG

SEQ ID NO: 60 - DNA, 48 bp, Artificial
  synthetic sequence
gccggcggcggcggatcaggcggcggcggatcaggcggcggcggatcc

SEQ ID NO: 61 - protein, 14 aa, Artificial
  synthetic sequence
GGGGSGGGGSGGGG

SEQ ID NO: 62 - DNA, 43 bp, Artificial
  synthetic sequence
ggacatatgctgtgatcattcccaaccattcccttatccaggc

SEQ ID NO: 63 - DNA, 41 bp, Artificial
  synthetic sequence
cgcgaattcgatccatggaagccacagctgccctccacaga

SEQ ID NO: 64 - DNA, 36 bp, Artificial
  synthetic sequence
cgcgtcgacctagaagccacagctgccctccacaga

SEQ ID NO: 65 - DNA, 602 bp, Artificial
  synthetic sequence
catatgctgtgatcattcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttccatggatcgaattc

SEQ ID NO: 66 - protein, 192 aa, Artificial
  synthetic sequence
SFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 67 - DNA, 600 bp, Artificial
  synthetic sequence
catatgctgtgatcattcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctaggtcgacgcg

SEQ ID NO: 68 - protein, 192 aa, Artificial; identical to SEQ ID NO: 66
  synthetic sequence

SEQ ID NO: 69 - DNA, 639 bp, Artificial
  synthetic sequence
catatgctgtgatcattcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttcggcggcggcggatcaggcggcggcggatcaggcggcggcggatccgaattc

SEQ ID NO: 70 - protein, 206 aa, Artificial
  synthetic sequence
SFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFGGGGSGGGGSGGGG

SEQ ID NO: 71 - DNA, 630 bp, Artificial
  synthetic sequence
catatgttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttcggcggcggcggatcaggcggcggcggatcaggcggcggcggatccgaattc

SEQ ID NO: 72 - protein, 206 aa, Artificial
  synthetic sequence
MFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFGGGGSGGGGSGGGG

SEQ ID NO: 73 - DNA, 1248 bp, Artificial
  synthetic sequence
tgatcattcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttcggcggcggcggatcaggcggcggcggatcaggcggcggcggatcattcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttcggcggcggcggatcaggcggcggcggatcaggcggcggcggatccgaattc

SEQ ID NO: 74 - protein, 412 aa, Artificial
  synthetic sequence
SFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFGGGGSGGGGSGGGGSFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFGGGGSGGGGSGGGG

SEQ ID NO: 75 - DNA, 2445 bp, Artificial
  synthetic sequence
catatgttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttcggcggcggcggatcaggcggcggcggatcaggcggcggcggatcattcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttcggcggcggcggatcaggcggcggcggatcaggcggcggcggatcattcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttcggcggcggcggatcaggcggcggcggatcaggcggcggcggatcattcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctaggtcgacgcg

SEQ ID NO: 76 - protein, 810 aa, Artificial
  synthetic sequence
MFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFGGGGSGGGGSGGGGSFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFGGGGSGGGGSGGGGSFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFGGGGSGGGGSGGGGSFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 77 - DNA, 593 bp, Artificial
  synthetic sequence
catatgttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttccatggatcgaattc

SEQ ID NO: 78 - protein, 192 aa, Artificial; identical to SEQ ID NO: 8
  synthetic sequence

SEQ ID NO: 79 - DNA, 592 bp, Artificial
  synthetic sequence
aagctttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttccatggatcgaattc

SEQ ID NO: 80 - protein, 191 aa, Artificial [sequence omitted]

SEQ ID NO: 81 - DNA, 587 bp, Artificial
  synthetic sequence
aagctttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctagggatcc

SEQ ID NO: 82 - protein, 191 aa, Artificial [sequence omitted]

SEQ ID NO: 83 - DNA, 1165 bp, Artificial
  synthetic sequence
aagctttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttcttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttccatggatcgaattc

SEQ ID NO: 84 - protein, 191 aa, Artificial [sequence omitted]

SEQ ID NO: 85 - DNA, 2307 bp, Artificial
  synthetic sequence
catatgttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttcttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttcttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttcttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctagggatcc

SEQ ID NO: 86 - protein, 192 aa, Artificial; identical to SEQ ID NO: 8
  synthetic sequence

(19) United States
(12) Patent Application Publication
(10) Pub. No.: US 2004/0059093 A1
(43) Pub. Date: Mar. 25, 2004

(54) Methods to construct multimeric DNA and polymeric protein sequences as direct fusions or with linkers

(76) Inventor: Stuart Bussell, Carlsbad, CA (US)

Correspondence Address: DAVID R PRESTON & ASSOCIATES, 12625 HIGH BLUFF DRIVE, SUITE 205, SAN DIEGO, CA, 92130, US

(21) Appl. No.: 10/621,693

(22) Filed: Jul. 16, 2003

Related U.S. Application Data
(60) Provisional application No. 60/396,466, filed on Jul. 16, 2002.

(30) Foreign Application Priority Data
Jul. 16, 2003 (WO) PCT/US03/22216

(51) Int. Cl. C07K 14/705; C07K 14/47; C12Q 1/68; C07H 21/04; C12P 19/34

(52) U.S. Cl. 530/350; 435/6; 435/91.2; 536/23.5

(57) ABSTRACT

Methods are disclosed that enable the construction of families of sequences comprising sequence repeats connected as direct fusions or with linkers. Families are constructed from a series of interchangeable cassettes and consist of related sequences that can be easily and efficiently polymerized to form multimers and polymers ranging from 1 to N, where N is theoretically any integer greater than one.

### CROSS REFERENCE TO RELATED APPLICATONS

This application claims priority to U.S. provisional application number US 60/396,466, filed Jul. 16, 2002, naming Stuart Bussell as inventor.

### SEQUENCE LISTING

A sequence listing is provided in electronic and printed form and as an appendix to this application.

### BACKGROUND

The present invention relates generally to recombinant DNA technology and recombinant protein expression, and more specifically, to constructs comprising repeat DNA sequences and to methods of making constructs comprising repeat DNA sequences, including constructs that encode polymer peptides and proteins, in which monomers are either fused directly or with linkers.

Recombinant proteins have become an important class of therapeutics and diagnostics since their introduction in the 1980s. The first recombinant protein therapeutics replaced products isolated from either animal or human tissue. For example, recombinant human growth hormone (recombinant human GH or rhGH) replaced material isolated from the pituitaries of human cadavers (Jorgenson, Endocrine reviews 12:189, 1991). The need arose because of the transmission of a rare fatal disease, called Creutzfeldt-Jakob disease (CJD), that is transmitted from impurities in pituitary derived hGH. The level of control possible with the recombinant version enabled production of drug certifiably free of known communicable agents.

Another example of an early recombinant protein is recombinant human insulin (rhI) (Chien, Drug Development and Industrial Pharmacy 22:753, 1996). In this case, the recombinant product replaced, or supplemented, insulin isolated from the pancreases from swine and cattle. The recombinant protein exactly matches the one found naturally in humans, in contrast with the animal versions that differ by one to three amino acids.

More recombinant protein therapeutics followed including interferons, interleukins, hematopoetic factors, monoclonal antibodies, and others.

In the diagnostic field, antibodies, both natural and engineered, are used to recognize and signal the presence of clinical markers. An advantage of engineered antibody fragments over full-length antibodies is that they are amenable to production in facile expression systems such as E. coli or P. pastoris (Pennell et al., Res Immunol 149:599, 1998).

Some of the in vivo characteristics of recombinant drugs are described by their pharmacokinetic parameters. The field of pharmacokinetics concerns itself with the absorption, distribution, metabolism, and excretion (ADME) of compounds delivered in vivo. Basically, pharmacokinetic parameters describe the concentration of a drug distributed throughout the body over time.

Generally, absorption of protein drugs requires delivery by injection. A body's natural barriers tend to prevent the absorption of intact proteins if any other routes of delivery are used. The digestion system breaks down proteins administered orally, while the body's various epidermal surfaces prevent absorption throughout the body.

Once injected, proteins tend to distribute throughout the circulatory system where they can react (part of metabolism) with other molecules or undergo excretion. Mathematical models, of varying complexity, are available to explain experimental measurements of drug concentrations as a function of time. One of the basic pharmacokinetic parameters is a drugs half-life, t_{1/2}, which is characteristic of the drug's duration in the bloodstream.

A key determinant to a protein's half-life in the blood is its size, and this is a result of elimination of proteins from the blood by glomerular filtration in the kidneys (Venkatachalam et al., Circulation Research 43:337, 1978). Basically, the filtration allows proteins smaller than 60 kilodaltons (kD), and other similarly sized molecules, to pass out of the blood, resulting in urinary excretion, while retaining larger ones. This has a major impact on the dosing regimen for a given protein. Proteins smaller than 60 kD tend to need daily, or more frequent, injections.

One strategy to minimize the discomfort and inconvenience of daily injections is to prolong the action of proteins once introduced in vivo. Two basic strategies are used. One involves the formulation of the protein into a slow release formulation (Putney et al., Nature Biotechnology 16:153, 1998). An example of this technique involves formulating proteins into a biocompatible polymer, poly lactic co-glycolytic acid (PLGA), that dissolves slowly over time, releasing protein during the dissolution process. Recombinant hGH is one protein successfully formulated this way (Johnson et al., Nature Medicine 2:795, 1996). A disadvantage of this technique that complicates its widespread application is the challenge of formulating and manufacturing each protein so that it is stable during processing and use. Furthermore, injections of PLGA formulated proteins can be uncomfortable.

The other strategy to prolong a protein's in vivo action involves modifying the protein so that it acts like a larger particle and is excreted more slowly through the kidneys. While prolonging the proteins in vivo residence, the modification must avoid adverse consequences such as immunogenicity, toxicity, unwanted changes to the molecules distribution, and unwanted changes to its activity.

A common technique in protein modification involves conjugating a native protein to polyethylene glycol (PEG) or another protein (Roberts et al., Adv Drug Deliv Rev 54:459, 2002). PEG molecules are manufactured at all ranges of molecular weights. They can be attached to reactive chemical groups compatible with chemical conjugation to proteins, and they are safe in vivo. Pegylated proteins have been approved for human use. Pegylated interferon is an example (Sharieff et al., Cleve Clin J Med 69:155, 2002). Pegylation effectively enhances the size of the resulting conjugate while avoiding immunogenicity or activity alterations. However, PEG has its own chemical and physical characteristics, and this can alter a conjugates ADME. For example, PEG alters the distribution of IL2 in such a way as to unacceptably increase its toxicity (Chen et al., The Journal of Pharmacology and Experimental Therapeutics 293:248, 2000). Also, the chemical conjugation is difficult to completely control, and any resulting conjugate is likely to be a mix of chemical species.

Another promising technique involves conjugating or fusing proteins to a carrier protein. There are many examples of chimeric molecules formed either through chemical reaction between the parent proteins or through the fusion of their gene sequences. In the case of fusion proteins, experience shows that the separate polypeptides constituting a fusion protein generally fold into their three dimensional conformation independently. In fact, often a recombinant protein that misfolds during expression in E. coli by itself will fold properly when fused to a protein that regularly folds correctly. Examples include fusions to commercially available proteins such as GST and NusA (see for example Novagen, Madison, Wis.).

One technique to make therapeutic fusion proteins is to fuse native therapeutics to human serum albumin (HSA) (U.S. Pat. No. 5,876,969). HSA is a 66 kD protein that is abundant in the human bloodstream. It is non-immunogenic and readily available. Potential problems include changed distribution of any resulting conjugate and the effect of HSA as it is shuttled into cells that normally do not contain it intracellularly.

Another technique is to make therapeutic homomultimer fusion proteins. In this case, the coding DNA sequence for a functional protein is connected to copies of itself. A dimer of superoxide dismutase (“SOD”) is disclosed in U.S. Pat. No. 5,084,390, whereby the hinge region of an immunoglobin joins two copies of the SOD monomer. The resulting dimer has an extended in vivo half-life. In another example, a dimer of erythropoietin is disclosed in U.S. Pat. No. 6,242,570.

Methods to manufacture highly polymerized sequences, for example polymers having greater than two units, have been developed in the field of artificial protein polymers. Lewis et al (Protein Expression and Purification 7:400, 1996) reveal a method utilizing compatible, but nonregenerable, overhang restriction sites that are engineered to allow the polymerization of a monomeric spider silk repeating sequence in a geometric fashion. In similar manner, Elmorani, et al. (Biochemical and Biophysical Research Communication 239:240, 1997) use compatible, but nonregenerable, blunt end restriction sites to produce a polymeric form of wheat gliadin.

The techniques disclosed in both cases are predicated on the presence of a pair of compatible, nonregenerable, restriction sites at the end of the polymerizing protein sequence. This requirement severely limits the number of sequences that are amenable to polymerization. Another disadvantage of currently available methods is that once a final polymeric sequence is generated, the researchers must employ additional steps to engineer it with the appropriate 5′ and 3′ sequences for expression.

### SUMMARY OF THE INVENTION

The present invention provides methods to easily and quickly generate multimers, such as dimers and higher order multimers, of DNA sequences and their open reading frame protein translations, resulting in constructs for the expression of proteins of greater molecular weight and valency. Methods are described whereby a sequence is attached to one or more versions of itself, either via a direct fusion or with a linker, where each version shares strong homology and is generally considered the same via its sequence and mode of action. In addition, the multimer is attached to terminal functional elements. The monomer can theoretically have any sequence and can consist of elements from one or more genes or synthetic DNA fragments. Thus, although the polymerization employs homomultimers, the fundamental monomers themselves can be generated from heterogeneous sequences. Furthermore, heteromultimers can be produced from monomers previously manipulated with the methods of this invention if the constitutive monomers have compatible ends.

In one aspect, the present invention comprises multimer assemblies of cassettes that comprise nucleic acid sequences having restriction sites that can be ligated together to form constructs (multimer cassettes) having multiple copies of a sequence of interest (the monomer sequence), such as a sequence that encodes a peptide or protein. Restriction sites used to ligate cassettes of a multimer assembly together to make a multimer cassette comprise restriction pair members that when ligated together, do not regenerate a restriction site. In one embodiment of the present invention, multimer assemblies are used that comprise 1) at least one amplification cassette comprising at least a monomer sequence and 2) at least one 3′-terminal cassette comprising at least one 3′ specific sequence or at least one 5′-terminal cassette comprising at least one 5′ specific sequence. Preferably, the 5′-terminal and/or 3′-terminal cassettes additionally comprise at least a portion of the monomer sequence.

In some preferred embodiments of this aspect of the invention, component cassettes (such as amplification cassettes, 5′-terminal and/or 3′-terminal cassettes) of a multimer assembly can comprise one or more flanking restriction sites that can facilitate cloning of multimer cassettes.

In some preferred embodiments, component cassettes (such as amplification cassettes, 5′-terminal and/or 3′-terminal cassettes) can comprise one or more linker sequences, such as linker sequences that encode amino acids or peptides that can be used to link monomers. Such linker sequence can also comprise restriction sites, such as restriction pair members that can be used in making multimer cassettes.

In another aspect, the present invention provides methods of making multimer cassettes. Such methods include ligation of 3′ and 5′ restriction pair members of component cassettes. In some preferred embodiments, the synthesis of multimer cassettes can optionally make use of flanking restriction sites that can be provided in the component cassettes. In some preferred embodiments, the synthesis of multimer cassettes can optionally make use of restriction sites that can be provided in linker sequences included in one or more component cassettes.

The protein polymers encoded by DNA multimers of a multimer cassette can be expressed in any suitable gene/protein expression system. For example, prokaryotic or eukaryotic systems are suitable, as are in vitro translation systems. The multimer assembly system described here facilitates the multimerization process and enables the production of multimers of any size and with a variety of N-terminal, linker, and C-terminal elements from a limited number of starting DNA sequences. For example, a gene can be designed for intracellular expression with an N-terminal methionine and for extracellular expression by including a secretory signal sequence after the N-terminal methionine.

The invention can be used to produce constructs having multimeric or polymeric sequences of increased size and multiplicity.

### BRIEF DESCRIPTION OF THE DRAWINGS

FIG. 1 is a diagram showing an example of a multimer assembly and its cassettes for monomers having a terminal restriction pair. (A) shows a 5′-terminal cassette with sequence elements coding for protein N-terminal elements. The crosshatched elements are restriction sites, the rectangular segments are portions of the monomer sequence, the looping arrows indicate continuation as a plasmid, straight arrows indicate linker sequences, and ˜ refers to arbitrary DNA sequences. The circle is a start codon, and the square is a 5′ specific sequence. Restriction site 1 can include the start codon and/or can be a flanking restriction site for cloning flexibility. Restriction site 3 is the 3′ restriction pair member, and 2 and 4 are flanking restriction sites for cloning flexibility. (B) shows an amplification cassette with sequence elements coding for a polymerizing sequence. Restriction site 5 is the 5′ restriction pair member. (C) shows a 3′-terminal cassette with sequence elements coding for C-terminal elements. The pentagon represents 3′ specific sequence and the hexagon a stop codon. The restriction site arrangement is preferred, but not the only arrangement for construction of an insert cassette. (D) shows one example of a Linker sequence. As shown here, it can contain elements 5′ and 3′ of the restriction pair formed by ligating restriction sites 5 and 3 together. The left and right arrows represent linker 5′ and 3′ elements, respectively.

FIG. 2 is a diagram showing one example of a multimer assembly and its cassettes for a monomer with an internal restriction pair. The crosshatched elements are restriction sites, the rectangular segments are portions of the monomer sequence, the looping arrows indicate continuation as a plasmid, straight arrows indicate linker sequences, and ˜ refers to arbitrary DNA sequences. The circle is a start codon, and the square is a 5′ specific sequence. The pentagon represents 3′ specific sequence and the hexagon a stop codon. (A) shows a 5′-terminal cassette with sequence elements coding for N-terminal elements. (B) shows an amplification cassette with sequence elements coding for the polymerizing sequence. The double arrow represents a linker (optional). (C) shows a 3′-terminal cassette with sequence elements coding for C-terminal elements. (D) shows an alternative 3′-terminal cassette that requires use of sequential ligation to form a multimer expression cassette.

FIG. 3 is a diagram showing two examples of pathways that can be used in the polymerization of amplification cassettes. Both procedures depicted involve two generalized cassettes, one with insert sequence b1 and the other with insert sequence b2. For pathway A, the b2 containing cassette is opened by digesting with enzymes 1 and 5. The b1 insert sequence is isolated after digesting the b1 containing cassette with enzymes 1 and 3. For pathway B, the b1 containing cassette is opened by digesting with enzymes 2 and 3. The b2 insert sequence is isolated after digesting the b2 containing cassette with enzymes 2 and 5. The final ligations to generate multimer assemblies are similar for both cases. The crosshatched elements are restriction sites, the rectangular segments are insert sequences, the looping arrows indicate continuation as a plasmid, and ˜ refers to arbitrary DNA sequences.

FIG. 4 is a diagram showing examples of sequential ligation of cassettes to create a functional multimer cassette of a desired size. The schematic is a generalization of the sequential ligation procedure necessary for use with a 3′-terminal cassette given in FIG. 2D. Pathway A depicts the insertion of an ‘S’ plasmid fragment into a ‘T’ containing plasmid, while Pathway B depicts the insertion of a ‘T’ plasmid fragment into a ‘S’ containing plasmid. In the figure, S+T=5I+AI, AI+3I, 5IAI+3I, or 5I+AI3I, where 5I≡the insert from a 5′-terminal cassette, AI≡the insert from an amplification cassette, 3I≡the insert from a 3′-terminal cassette, 5IAI≡the insert resulting from the ligation of 5I and AI, AI3I≡the insert resulting from the ligation of AI with 3I, and 5IAI3I=≡the insert resulting from the ligation of 5I with AI3I or 5IAI with 3I. Formation of 5IAI3I requires two sequential ligations and generation of intermediate 5IAI or AI3I cassettes for each polymer size made. The crosshatched elements are restriction sites, the rectangular segments are insert sequences, the looping arrows indicate continuation as a plasmid, and ˜ refers to arbitrary DNA sequences.

FIG. 5 is a diagram showing possible methods for generation of an insertion cassette. Pathways A and B are alternative pathways for insertion cassette generation based on different arrangements of flanking restriction sites. Pathway A involves opening the 5′-terminal cassette and inserting a fragment from the 3′-terminal cassette, while Pathway B involves opening the 3′-terminal cassette and inserting a fragment from the 5′-terminal cassette. The crosshatched elements are restriction sites, the rectangular segments are portions of the monomer sequence, the looping arrows indicate continuation as a plasmid, straight arrows indicate linker sequences, and ˜ refers to arbitrary DNA sequences. The circle is a start codon, and the square is a 5′ specific sequence. The pentagon represents 3′ specific sequence and the hexagon a stop codon.

FIG. 6 is a diagram showing one possible method of generating a functional multimer cassette of a desired size from an insertion cassette and an amplification cassette. The insertion cassette is opened at both sites of the restriction pair with subsequent ligation of the insert from an amplification cassette, but the insert can ligate in the wrong orientation. Correct inserts must be identified by subsequent analysis. The crosshatched elements are restriction sites, the rectangular segments are portions of the monomer sequence, the looping arrows indicate continuation as a plasmid, straight arrows indicate linker sequences, and ˜ refers to arbitrary DNA sequences. The circle is a start codon, and the square is a 5′ specific sequence. The pentagon represents 3′ specific sequence and the hexagon a stop codon.

FIG. 7 is a diagram showing another possible method of generating a functional multimer cassette of a desired size from an insertion cassette and an amplification cassette. The insertion cassette is opened with enzymes 3 and 2 to create an oriented ligation, but an additional step is required. In this case, the amplification cassette has flanking restriction site 2 on the 3′ side of restriction site 3. The crosshatched elements are restriction sites, the rectangular segments are portions of the monomer sequence, the looping arrows indicate continuation as a plasmid, straight arrows indicate linker sequences, and ˜ refers to arbitrary DNA sequences. The circle is a start codon, and the square is a 5′ specific sequence. The pentagon represents 3′ specific sequence and the hexagon a stop codon.

FIG. 8 is a diagram showing another possible scheme for generating a functional multimer cassette of a desired size from an insertion cassette and an amplification cassette in similar fashion to FIG. 7, but the amplification cassette has flanking restriction site 2 on the 5′ side of restriction site 5.

FIG. 9 is a diagram showing the PCR amplification of the hGH gene, its subsequent ligation to generate p0A0, and the ligation of the OmpA leader sequence to generate p0C0A2.

FIG. 10 is a diagram showing the PCR mutagenesis of the hGH gene to generate p0A01. The diagram also shows the ligation of the OmpA sequence into p0A01 to generate p0A11A2 and the ligation of the PstI/BamHI fragment from p0A01 into P0A03 to generate p0A11A1.

FIG. 11 is a diagram showing the PCR mutagenesis of the hGH gene to generate p0A11B.

FIG. 12 is a diagram showing the ligation of synthetic sequences to generate p0A11C1 and p0A11C2.

FIG. 13 is diagram showing the polymerization of a GH direct fusion amplification cassette.

FIG. 14 is diagram showing the generation of the GH direct fusion insertion cassette, p0A11D, and subsequent ligation of an amplification cassette to generate a multimer expression cassette.

FIG. 15 is a diagram showing the PCR mutagenesis of the hGH gene to generate p0A21B, the base amplification cassette for the GH glycine linker assembly.

FIG. 16 is a diagram showing the PCR mutagenesis of the hGH gene to generate the base cassettes, p0A31A, p0A31B, and p0A31C, for the GH SWG₄S assembly.

FIG. 17 is a diagram showing the sequential ligation of the GH SWG₄S assembly cassettes to generate the multimer expression cassette, p0A31E3.

FIG. 18 is a picture of an SDS-PAGE gel showing the separation of proteins by molecular weight from separate lysates from cells expressing different polymers of rhGH. Lane 1 contains molecular weight standards, lane 2 the rhGH monomer, lane 3 the rhGH dimer, lane 4 the rhGH trimer, lane 5 the rhGH pentamer, and lane 6 the rhGH nanamer.

FIG. 19 is a diagram showing insertion of synthetic sequences to generate the G₄S assembly 5′-terminal and amplification cassettes.

FIG. 20 is a diagram showing PCR mutagenesis of the hGH gene to generate p0A04 and p0A41C.

FIG. 21 is a diagram showing ligation of the insert from p0D13A with p0A04 to generate p0A43B and ligation of the PstI/EcoRI fragment from p0A11A1 to generate p0A43A.

FIG. 22 is a diagram showing ligations to generate the base cassettes; p0A51A, p0A51B, and p0A51C, for the GH direct fusion assembly utilizing blunt ended HindIII and NcoI sites for the restriction pair.

FIG. 23 is a diagram showing the polymerization of the p0A51B insert to generate p0A51B2.

### DETAILED DESCRIPTION OF THE INVENTION

### Introduction

The current invention discloses methods that extend the polymerization techniques in three important ways. First, it introduces new methods to generate highly polymerized sequences from monomers that are incompatible with previous protein polymerization techniques. Second, it introduces additional linker sequences that, when paired with the monomer sequences, facilitate their use. Third, it introduces methods that facilitate the construction and expression of functional multimers and polymers. Taken together, the new methods enable the generation of large numbers of polymer variants that can differ in sequence and degree of polymerization. These variants can then be tested for desirable traits.

The disclosed techniques are applicable to any polypeptide sequence and can prove useful for proteins for which increased total molecular weight is deemed advantageous. The disclosed techniques are also useful for proteins for which increased valency is deemed advantageous. For example, expression of single chain antibody fragments fused together as larger multimers have the advantage of high valency and a stable linkage. Furthermore, if cassettes for two different sequences share compatible restriction pair members, they can be co-polymerized to produce heteromultimers.

### Definitions

Unless defined otherwise, all technical and scientific terms used herein have the same meaning as commonly understood by one of ordinary skill in the art to which this invention belongs. Conventional methods are used for these procedures, such as those provided in the art and various general references. Where a term is provided in the singular, the inventors also contemplate the plural of that term. The nomenclature used herein and the laboratory procedures described below are those well known and commonly employed in the art. Where there are discrepancies in terms and definitions used in references that are incorporated by reference, the terms used in this invention shall have the definitions given herein. As employed throughout the disclosure, the following terms, unless otherwise indicated, shall be understood to have the following meanings:

Monomer. A DNA or amino acid sequence whose polymerization is desirable. A monomer can be a portion of a naturally occurring sequence (for example, a binding domain of an antibody). The sequence can be derived from one or more naturally occurring ones, or can be a synthetic sequence, or can be any combination of sequences of synthetic and natural origins. Monomers of the present invention can comprise linkers. As used herein monomer sequence means a nucleic acid sequence.

Multimer. A nucleic acid sequence encoding two or more monomers.

Polymer or Multimeric protein. A functional polypeptide that can be synthesized from a multimer assembly of the present invention. A polymer comprises at least two monomers (where each monomer can optionally comprise one or more linkers), can comprise one or more 5′ translated regions (for example, signal peptides, N-terminal regions, “pro” or “pre” protein sequences, tag sequences, etc.), and can comprise one or more 3′-translated regions (for example, C-terminal regions; tag sequences, etc.)

Linker. A linker is a DNA or amino acid sequence that connects one DNA sequence with another through covalent bonds or an amino acid or peptide that connects one peptide or protein unit with another peptide or protein unit through peptide bonds. An amino acid or peptide linker can be a single amino acid (for example, glycine) or can be more than one amino acid.

Restriction Pair. Two restriction sites that have different recognition sequences that are ligation compatible, but when ligated together do not regenerate either of the two original restriction sites. A restriction pair can include two restriction sites that have overhangs, such as BglII and BamHI, or can include any two blunt end restriction sites that do not have the same recognition sequence, such as StuI and NaeI. In a broader application, a restriction pair can also include restriction sites that are initially ligation incompatible but are blunt ended to make them ligation compatible. An example includes blunt ending HindIII and NcoI to make them ligation compatible.

Restriction pair member or restriction member. A restriction site that is part of a restriction pair. The 5′ and 3′ restriction pair members together make up a restriction pair, and each is the other's partner.

5′ restriction pair member or 5′ restriction member or 5′ member. A restriction pair member that is located at the 5′ terminus of a DNA sequence, such as a DNA sequence that, at least in part, encodes a monomer whose multimerization is desired or multimer of the present invention, or is located at the 5′ terminus of a DNA sequence of interest whose ligation to a multimer is desired. The term “5′ restriction pair member” or “5′ member” can be used to refer to an unaltered restriction site (for example, a Bam HI site) or to a restriction site that has been altered, such as, for example, a filled-in 5′ restriction pair member (such as blunt ended Bam HI site), or a fused 5′ restriction pair member (for example, a ligated BamHI/BglII site).

3′ restriction pair member or 3′ restriction member or 3′ member. A restriction pair member that is located at the 3′ terminus of a DNA sequence, such as a DNA sequence that, at least in part, encodes a monomer whose multimerization is desired or multimer of the present invention, or is located at the 3′ terminus of a DNA sequence of interest whose ligation to a multimer is desired. The term “3′ restriction pair member” or “3′ member” can be used to refer to an unaltered restriction site (for example, a BglII site) or to a restriction site that has been altered, such as, for example, a filled-in 3′ restriction pair member (such as blunt ended BglII site), or a fused 3′ restriction pair member (for example, a ligated BamHI/BglII site).

Flanking restriction site or flanking site. A restriction site that is not a member of a restriction pair used in the constructs and methods of the present invention. Its location outside of insert sequences and restriction pair members used in the cassettes and methods of the present invention can facilitate manipulation of the insert.

Insertion restriction site. A specific flanking restriction site that is 3′ of the 3′ restriction pair member of the 5′-terminal cassette and 5′ of the 5′ restriction pair member of the 3′-terminal cassette.

Amplification cassette. A DNA sequence that includes at least one monomer that is flanked by a restriction pair. An amplification cassette has a 5′ restriction pair member at its 5′ terminus and a 3′ restriction pair member at its 3′ terminus. The restriction pair enables the multimerization of the sequence or the ligation of it to other sequences with ligation compatible restriction sites. An amplification cassette can optionally comprise other sequences as well, such as but not limited to sequences that code for amino acid or peptide linkers.

5′-terminal cassette. A DNA sequence that comprises a 3′ restriction pair member, at least one 5′-specific sequence, where a 5′-specific sequence is a sequence that, when positioned at the 5′ end of a multimer sequence, can facilitate the use of DNA multimers or the expression, purification, or identification of at least one protein polymer of the present invention, and, preferably, at least a portion of a monomer sequence. The 3′ restriction pair member is ligation compatible with the 5′ terminus of at least one amplification cassette. The 5′-terminal cassette is useful for introducing 5′-terminal DNA sequences that contribute to making a sequence functional. Examples of 5′ specific sequences include, but are not limited to, the translation start codon, secretion sequences, tag sequences, linker sequences, or special restriction sites.

3′-terminal cassette. A DNA sequence that comprises a 5′ restriction pair member, at least one 3′-specific sequence, where a 3′-specific sequence is a sequence that, when positioned at the 3′ end of a multimer sequence, can facilitate the use of DNA multimers or the expression, purification, or identification of at least one protein polymer of the present invention, and, preferably, at least a portion of a monomer sequence. The 5′ restriction pair member is ligation compatible with the 3′ terminus of at least one amplification cassette. The 3′-terminal cassette is useful for introducing 3′-terminal DNA sequences that contribute to making a sequence functional. Examples of 3′ specific sequences include, but are not limited to, tag sequences, C-terminal sequences, polyadenylation sequences, stop codons, linker sequences, and the like.

Insert sequence. The functional sequence in a cassette. For the amplification cassette, the functional sequence includes both restriction pair members and all sequence in between, including the monomer sequence. For the 5′-terminal cassette, the functional sequence includes the 3′ restriction pair member, all 5′-specific sequences, and its portion of a monomer sequence, if present. For the 3′-terminal cassette, the functional sequence includes the 5′ restriction pair member, all 3′-specific sequences, and its portion of a monomer sequence, if present. For multimer cassettes, the functional sequence includes the functional sequences of the constitutive cassettes.

Multimer assembly. The collection of all cassettes that, in combination, after ligation, yields functional multimer DNA sequences or polymer protein sequences of a starting monomer. A multimer assembly comprises one or more 5′-terminal cassettes and one or more amplification cassettes; one or more amplification cassettes and one or more 3′-terminal cassettes; or one or more 5′-terminal cassettes, one or more amplification cassettes, and one or more 3′-terminal cassettes that can be fused using 3′ and 5′ restriction pair members.

Multimer cassette. A cassette resulting from the ligation of two or more cassettes from the same multimer assembly.

Insertion Cassette. A multimer cassette generated from the ligation of a 5′-terminal and 3′-terminal cassette of a multimer assembly that is ligation compatible with any of said assembly's amplification cassettes to generate a multimer cassette.

Multimer expression cassette. A multimer cassette that, when transcribed and translated in a suitable expression system, produces a polymer protein sequence of a starting monomer.

Segment of a monomer sequence. A segment of a monomer sequence is a portion of monomer sequence, that is, a nucleic acid sequence that encodes a portion of a monomer.

### I. Methods of Making Multimer Assemblies

The present invention includes methods of fusing two or more nucleic acid sequences. The nucleic acid sequences can encode for peptide or protein sequences, such that when the nucleic acid sequences are expressed, a polymeric protein is produced. Preferably, in the methods of the present invention, the peptide or protein monomers encoded by the nucleic acid sequences are identical peptide or protein monomers. However, this is not a requirement of the present invention. The nucleic acid sequence, whose polymerization is desired is called a monomer sequence.

Monomer sequences can encode proteins or peptides whose function is known or unknown. Preferably, however, the identity and function of the peptide or protein encoded by a monomer sequence is known. Of particular interest are peptides and proteins that can have diagnostic or therapeutic value (for example, human growth hormone, hGH), although the invention is not limited to these protein sequences.

For example, monomer sequences can encode at least a portion of one or more receptors, receptor ligands, enzymes, inhibitors, transcription factors, translation factors, DNA replication factors, activators, chaperonins, or antibodies. Monomer sequences can also encode at least a portion of one or more cytokines, growth factors, or hormones such as, but not limited to, Interferon-alpha, Interferon-beta, Interferon-gamma, Interleukin-1, Interleukin-2, Interleukin-3, Interleukin-4, Interleukin-5, Interleukin-6, Interleukin-7, Interleukin-8, Interleukin-9, Interleukin-10, Interleukin-11, Interleukin-12, Interleukin-13, Interleukin-14, Interleukin-15, Interleukin-16, Erythropoietin, Colony-Stimulating Factor-1, Granulocyte Colony-stimulating Factor, Granulocyte-Macrophage Colony-Stimulating Factor, Leukemia Inhibitory Factor, Tumor Necrosis Factor, Lymphotoxin, Platelet-Derived Growth Factor, Fibroblast Growth Factors, Vascular Endothelial Cell Growth Factor, Epidermal Growth Factor, Transforming Growth Factor-beta, Transforming Growth Factor-alpha, Thrombopoietin, Stem Cell Factor, Oncostatin M, Amphiregulin, Mullerian-Inhibiting Substance, B-Cell Growth Factor, Macrophage Migration Inhibiting Factor, Endostatin, and Angiostatin. Descriptions of these proteins can be found in Human Cytokines: Handbook for Basic and Clinical Research, Aggarwal, B. B. and Gutterman, J. U. Eds., Blackwell Scientific Publications, Boston, Mass., (1992), which is herein incorporated by reference in its entirety.

The monomer encoding sequences are polymerized together by ligation of compatible, nonregenerable restriction sites, called restriction pair members. Unlike previous methodologies, the present invention employs cassettes with sequences other than those encoding the original monomer itself in the construction process. For example:

In the methods of the present invention, multimer assemblies are used that comprise at least one amplification cassette and at least one of the following: at least one 3′-terminal cassette or at least one 5′-terminal cassette. An amplification cassette comprises an insert sequence that includes a monomer sequence whose polymerization is desired, a 5′ restriction pair member at its 5′ terminus, and a 3′ restriction pair member at its 3′ terminus. A 3′-terminal cassette comprises an insert sequence that includes at least one 3′ specific sequence and a 5′ restriction pair member site that can be fused to a 3′ restriction pair member site of at least one of the one or more amplification cassettes. A 5′-terminal cassette, comprises an insert sequence that includes at least one 5′ specific sequence and a 3′ restriction pair member site that can be fused to a 5′ restriction pair member site of at least one of the one or more amplification cassettes. Preferably, the 5′-terminal and/or 3′-terminal cassettes additionally comprise at least a portion of the monomer sequence.

5′ specific sequences can be, but are not limited to, sequences that enhance transcription, translation, secretion, protein folding, protein solubility, or binding of the protein to specific binding members such as antibodies. 3′ specific sequences can be, but are not limited to, stop codons or sequences that enhance RNA stability, protein folding, protein solubility, or binding of the protein to specific binding members such as antibodies.

In the multimer assemblies of the present invention, 5′ and 3′ restriction pair members are used to fuse amplification cassettes, and preferably, where applicable, 3′-terminal cassettes to amplification cassettes and 5′-terminal cassettes to amplification cassettes. 5′ and 3′ restriction pair members are preferably unique restriction sites that are ligation compatible, and said ligation destroys each member. In the alternative, 5′ and 3′ restriction pair members can be ligation incompatible sites that are made ligation compatible by blunt ending.

One aspect of the present invention is construction of cassettes comprising one or more flanking restriction sites that aid their use, but this is not a requirement of the present invention. Preferably, 3′-terminal cassettes and 5′-terminal cassettes, if present, comprise 3′ and 5′ flanking restriction sites. Flanking restriction sites can be any restriction site (except restriction pair member sites used in the same construct), and preferably aid the use of cassettes by increasing the facility of making multimer cassettes. For example, the flanking sites facilitate the manipulation of the insert sequences, including their isolation and ligation. For example, some preferred methods employ an insertion restriction site, which is a specific flanking restriction site that is 3′ of the 3′ restriction pair member of the 5′-terminal cassette and 5′ of the 5′ restriction pair member of the 3′-terminal cassette. Flanking restriction sites can also optionally be used to transfer constructs and assemblies to different expression vectors

In some preferred methods of the invention, sequences encoding linkers are employed. Multimer assembly cassettes can comprise one or more linker sequences. Multimer assembly cassettes can have linker sequences 5′ of one or more insert sequences, 3′ of one or more insert sequences, or both 5′ and 3′ of one or more insert sequences. Linker sequences can be part of amplification cassettes, 5′-terminal cassettes, 3′-terminal cassettes, or any combination thereof. In preferred aspects of the present invention, nucleic acid sequences that encode amino acid or peptide linkers that are used to link monomers can also comprise restriction sites, such as 3′ or 5′ restriction pair member sites that can facilitate construction of multimer assemblies. This provides a convenient means for introducing restriction pair members for efficient polymerization of monomer sequences through amplification cassettes and optionally 5′-terminal cassette or 3′-terminal cassette ligations. Alternatively, or in addition, amino acid or peptide linkers can be used to provide optimal spacing or folding of translated monomers or a polymer.

Where more than one linker sequence is used in a single multimer assembly cassette, they may or may not occur between each and every monomer sequence. Where more than one linker sequence is used in a single multimer assembly cassette, they can encode the same or different amino acid or peptide linkers.

Peptide linkers are well known in the art. Preferably linkers are between one and twenty amino acids in length, and more preferably between one and ten amino acids in length, although length is not a limitation in the linkers of the present invention. Preferably linkers comprise amino acid sequences that do not interfere with the conformation and activity of peptides or proteins encoded by monomers of the present invention. Some preferred linkers of the present invention are those that include the amino acid glycine. Examples include those disclosed in Table 1.

In an expressed protein polymer, such amino acid or peptide sequences join peptide or protein monomer sequences. If a linker is part of the insert sequence of the amplification cassette, it becomes part of the monomer that is to be multimerized. The linker sequence can comprise at least one restriction pair member.

The present invention also introduces several methods to expand the use of restriction pair member sites. For example:

In some methods of the present invention, restriction pair members that are used to join monomer sequences are internal to a monomer sequence. In these embodiments, an amplification cassette comprises a 5′ segment of a monomer sequence and a 3′ segment of a monomer sequence that together comprise the sequence of a complete monomer. The 5′ segment is positioned 3′ of the 3′ segment, the 5′terminus of the 3′ segment is a 5′ restriction pair member, and the 3′ terminus of the 5′ segment is a 3′ restriction pair member. In this case, in making a multimer cassette, ligation of the 3′ restriction pair member of the 5′ segment of one amplification cassette with the 5′ restriction pair member of the 3′ segment of another amplification cassette can form a complete monomer sequence. In order to complete the polymer sequences, a multimer assembly preferably comprises a 5′-terminal cassette that comprises the 5′ monomer segment and a 3′-terminal cassette that comprises the 3′ monomer segment. In this way, monomer sequences provided in the amplification cassettes can be provided in non-contiguous segments. In some preferred methods of the present invention, the amplification cassette further comprises a linker that is positioned between the 5′ segment and the 3′ segment of the monomer sequence.

In some methods of the present invention, restriction pair members can be overhang restriction sites. In some methods of the present invention, restriction pair members can be blunt end restriction sites. In some other methods of the present invention, restriction pair members are incompatible “overhang” restriction sites that are converted to blunt end restriction sites through the use of polymerases or nucleases.

In some preferred methods of the present invention, restriction pair members are conveniently provided in one or more linker sequences. In these embodiments, linker sequences comprising a restriction pair member can be engineered onto the 3′, 5′, or both ends of an insert sequence.

In some preferred methods of the present invention, the 3′-restriction pair member codes for a stop codon that is destroyed upon ligation to the 5′-restriction pair member.

In one aspect of the present invention, the assembly methodology consists of the following four steps:

1. Generate or Obtain the DNA for the Monomer.

Techniques familiar to those skilled in the art include, but are not limited to:

a. Amplification of a sequence from a DNA library, optionally including any additions or mutations to the sequence in PCR primers.

b. Chemical synthesis of the sequence

c. Splicing of sequences together from pre-existing DNA

2. Decide What Linker Sequence, if any, to Use Between Monomers and Construct a Multimer Assembly.

Options for the linker include none (direct fusion of monomers), a linker encompassing a restriction pair member within its sequence, a linker with restriction pair members at one or more termini, or a linker lacking a restriction pair member. Once a linker is added, it becomes part of the monomer sequence.

For each option, three basic cassettes can be generated: one or more 5′-terminal cassettes, at least one amplification cassette, and one or more 3′-terminal cassettes. However, in some instances, all three cassettes are not required. A multimer assembly comprises at least one amplification cassette, and one or more 5′-terminal cassettes or one or more 3′-terminal cassettes, or can have at least one amplification cassette, one or more 5′-terminal cassettes, and one or more 3′-terminal cassettes. In some cases, multiple versions of each cassette may be desirable. Furthermore, the amplification cassette can be polymerized to produce new higher order (multimeric) amplification cassettes.

The ends of the monomers determine the characteristics of the cassettes. The current invention discloses the use of linkers to introduce ends containing a restriction pair as well the construction of 5′-terminal and/or 3′-terminal cassettes to facilitate their use.

As an alternative to engineering the ends of a monomer with a restriction pair, then the cassettes can be constructed with a restriction pair internal to the monomer sequence. The construction of the cassettes is modified to accommodate the presence of an noncontiguous monomer in each.

Finally, a method is disclosed in which the constructions for a restriction pair either at the ends or internal to the monomer is extended to use with a pair of incompatible restriction sites. This method is less preferred, as the method requires that blunt ends for ligation are created for each ligation step (by nuclease digestion or polymerase fill-in, or both), decreasing the efficiency of the procedure.

The following are the general steps for construction of the assemblies for each possible restriction pair case:

a. Using a monomer sequence with a terminal restriction pair.

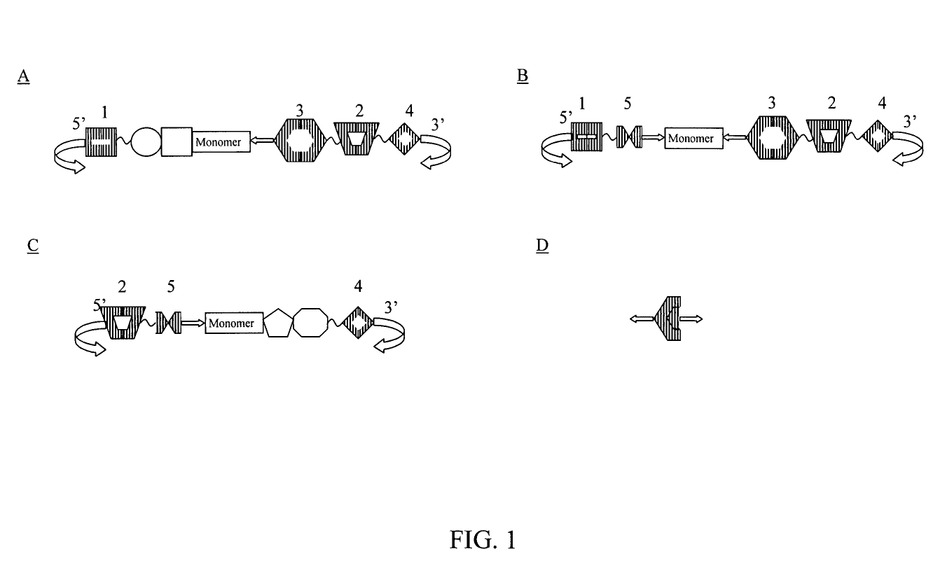

The scheme shown in FIG. 1 is applicable for any monomer sequence that can be engineered with a terminal restriction pair. The steps to engineer the assembly can include the following:

(1) Engineer 5′-terminal cassettes containing one or more 5′ specific DNA sequences (for example, start codon, secretion sequence, etc.), preferably the monomer sequence, linker sequence, if present, and the 3′ member of the restriction pair.

(2) Engineer an amplification cassette containing a 5′ restriction member, optionally a first linker sequence, at least one monomer sequence, optionally a second linker sequence, and a 3′ restriction member.

(3) Engineer 3′-terminal cassettes containing a 5′ restriction member, optionally a linker sequence, preferably the monomer sequence, and one or more 3′-terminal specific DNA sequences (specific recognition sequences, stop codon, etc.).

An alternative formulation involves 5′-terminal and/or 3′-terminal cassettes that do not include any monomer sequence. The utility of including the monomer sequence in both terminal cassettes lies in utilizing the restriction pair members to join each terminal cassette to an amplification cassette, however, this is not a requirement of the present invention.

b. Using a monomer sequence with an internal restriction pair.

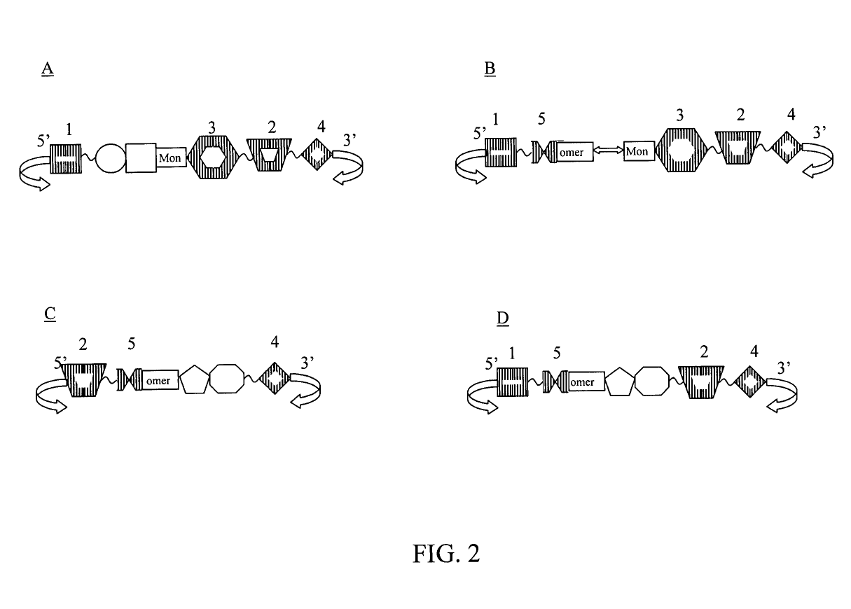

The scheme shown in FIG. 2 is applicable for any monomer sequence that can be engineered with an internal restriction pair. The steps to engineer the assembly include the following:

(1) Engineer 5′-terminal cassettes containing one or more 5′ specific DNA sequences (start codon, secretion sequence, etc.), the portion of a monomer sequence that occurs on the 5′ side of the restriction pair (the 5′ monomer segment), and finally the 3′ restriction pair member.

(2) Engineer an amplification cassette containing a 5′ restriction pair member, DNA encoding the portion of a monomer sequence that occurs 3′ of the restriction pair (the 3′ monomer segment), optionally a linker sequence, DNA encoding the portion of a monomer that occurs 5′ of the restriction pair (the 5′ monomer segment), and a 3′ restriction pair member.

(3) Engineer 3′-terminal cassettes containing the 5′ restriction pair member, the portion of a monomer sequence that occurs 3′ of the restriction pair (the 3′ monomer segment), and one or more 3′-terminal specific DNA sequences (specific recognition sequences, stop codon, etc.).

c. Using a monomer sequence with a pair of incompatible restriction sites made compatible by blunt ending.

Either scheme shown in FIG. 1 or FIG. 2 are applicable, but in this case the restriction pair consists of restriction sites that are blunt ended to make them compatible.

Once constructed, the amplification cassette enables generation of a sequence containing any number of monomers fused together.

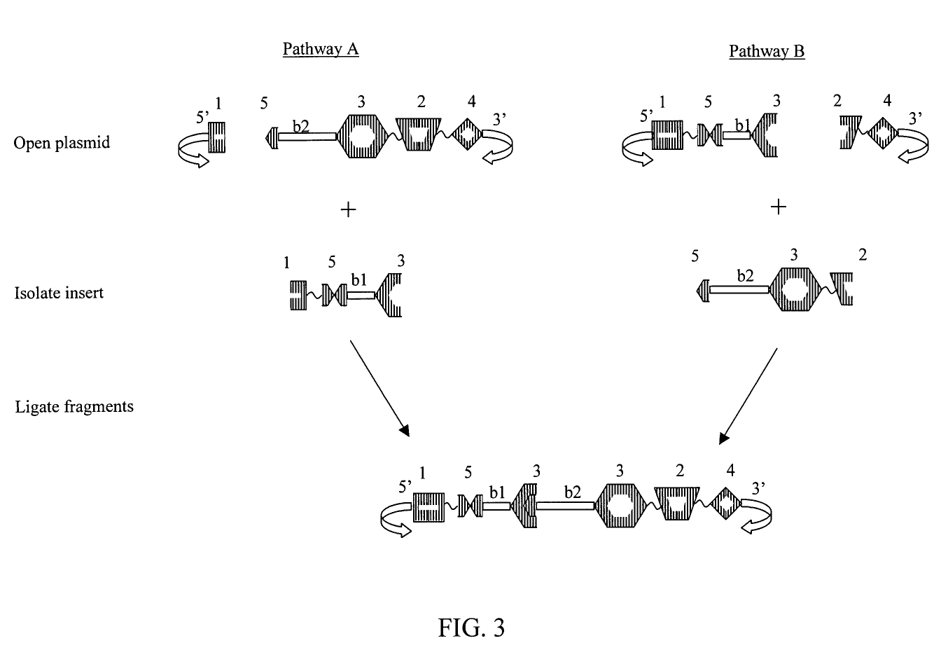

3. Polymerize the Amplification Cassette in an Arithmetic, Geometric, or Mixed Progression (see FIG. 3).

A series of amplification cassettes are generated from the original amplification cassette. The technique involves digesting a first construct comprising an amplification cassette at two 5′ or two 3′ sites of an insert, one of which is a restriction pair member site and the other of which is an external flanking site (external to the restriction pair member site), to open up the construct. This is followed by digesting a second construct comprising an amplification cassette at the same flanking site, but with the opposite restriction pair member, to release the amplification sequence from the plasmid as a fragment. This sequence is then ligated into the opened first plasmid construct from before. Both restriction sites used in the ligation are destroyed, but the resulting cassette has intact flanking restriction sites and an intact restriction pair on the ends that enable further polymerizations.

Mixing and matching the cassettes used to open a construct that comprises an amplification cassette and to generate an insert from a construct that comprises an amplification cassette enables new cassettes of any size to be made in an arithmetic, geometric, or mixed progression. For example, if the monomer is used to both open the plasmid and create insert, a dimer cassette is made. If the resulting dimer is used for both, then a tetramer is made. If this tetramer is used for both, then an octamer is made, and continuation leads to a binomial geometric progression. On the other hand, if the monomer is always used as the insert and the newest cassette is used to receive the insert, an arithmetic progression of one is produced. For instance, when a dimer construct is opened and a monomer fragment inserted, then a trimer is produced. When a trimer construct is opened and a monomer fragment is inserted, then a tetramer is produced. In general, any new cassette can be mixed with any previously generated cassette to allow rapid generation of a polymer of any desired size. For example, if a polymer of size 20 is desired, the 16 mer is generated geometrically, and ligating the 16 mer to the tetramer generates the 20 mer in a total of only 5 ligations.

Subsequent ligation to 5′- and 3′-terminal cassettes can enable production of a functional multimer. The multimer's size, based on actual molecular weight, is approximately a whole number multiple of the original. In addition, the composition of the multimer is almost identical to the monomer, differing only because of any linker sequences or terminal flanking regions that are used.

It is important to note that the polymerization does not require flanking sites. Without flanking sites, the ligations can occur with the fragments joined in either orientation, and more laborious subsequent analysis is needed to identify the correct constructs. In contrast, use of flanking sites facilitates the process by enabling oriented ligations.

4. Ligate the Cassettes Together to Give a Full Length, Functional, Multimer.

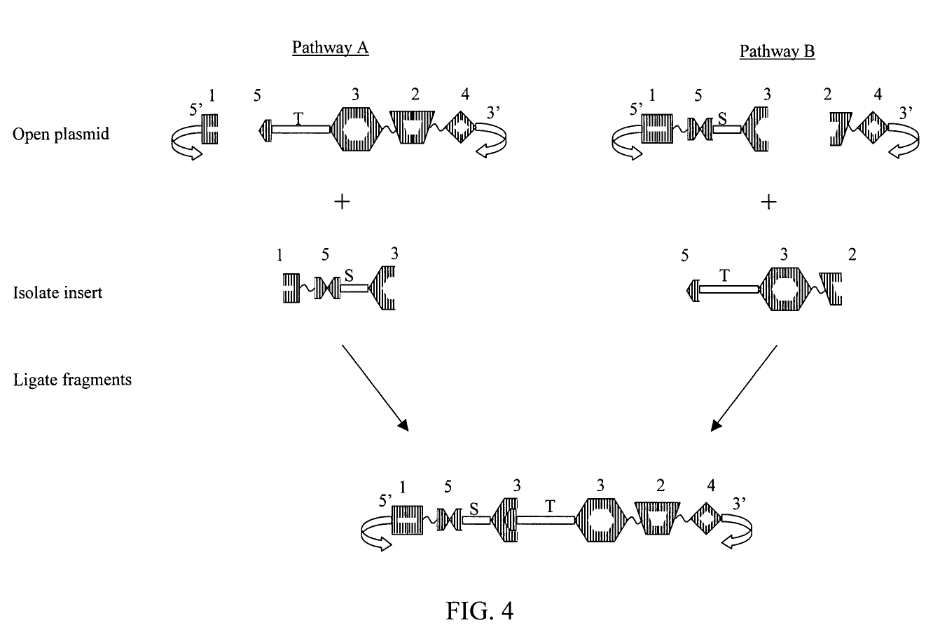

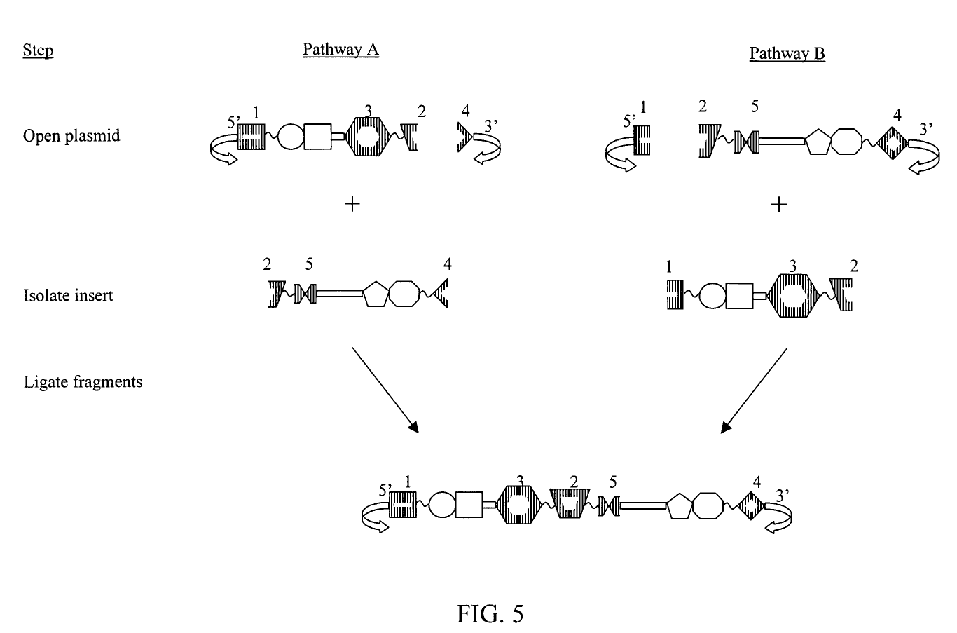

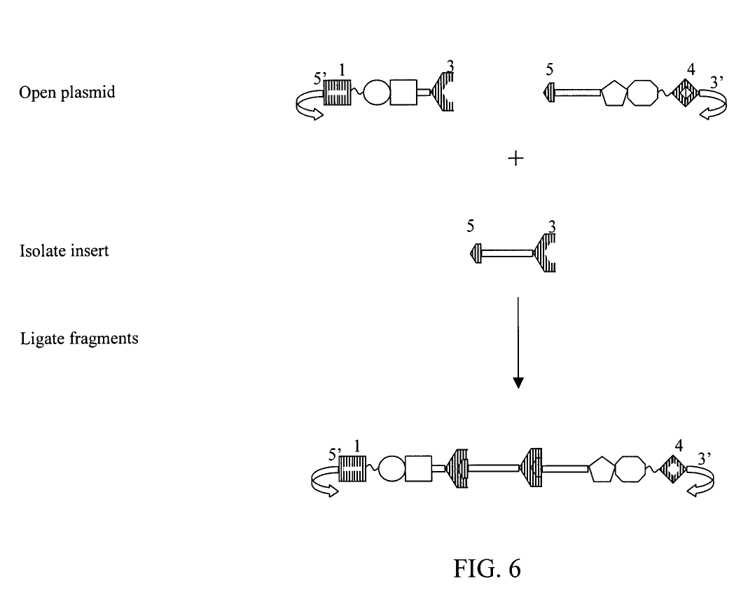

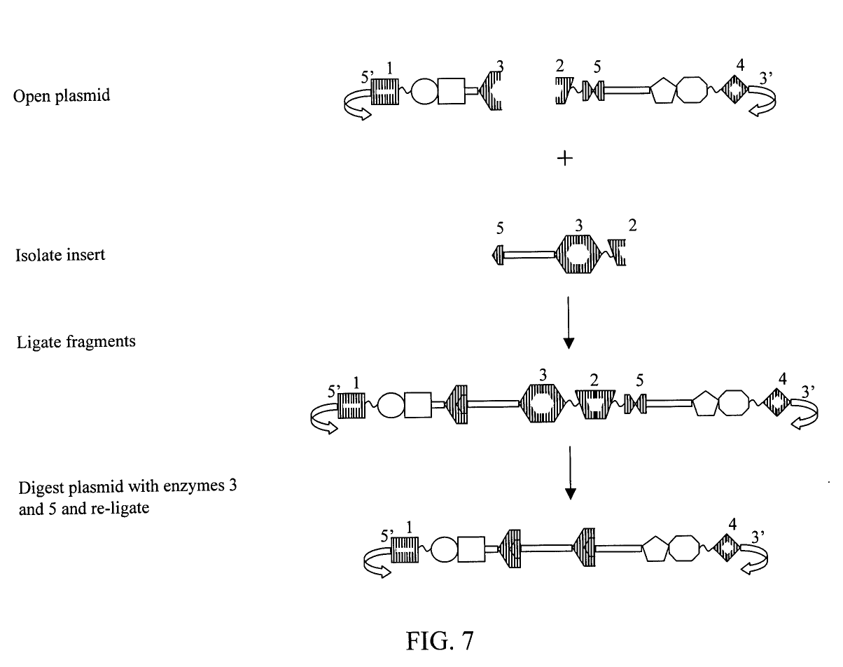

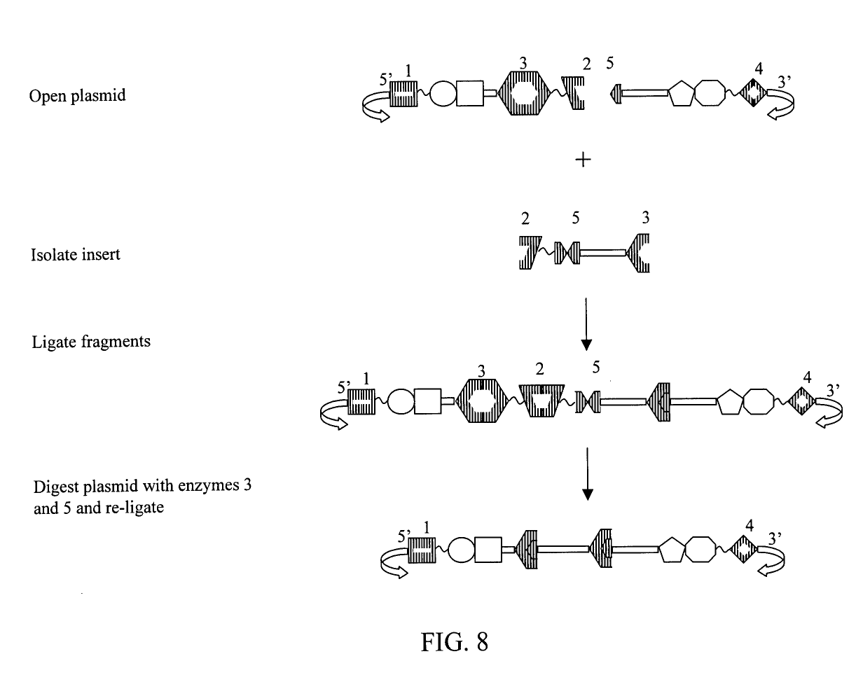

The cassettes can be ligated sequentially as shown in FIG. 4, or an insertion cassette can be created from the 5′- and 3′-terminal cassettes as diagramed in FIG. 5 with subsequent insertion of the polymerized amplification cassette as shown in FIGS. 6, 7, and 8. The use of an insertion cassette expedites the creation of a series of multimers with the same 5′ and 3′ terminal elements. FIG. 6 illustrates a technique for the ligation of the fragment from an amplification cassette into an insertion cassette using only the restriction pair restriction sites. However, the ligation is not oriented, necessitating additional analysis to identify correct constructs. FIGS. 7 and 8 show equivalent oriented ligations that result from different arrangements of flanking sequences.

FIG. 4 illustrates a method of making a multimer cassette from two cassettes from a multimer assembly utilizing flanking sites comprising a first cassette comprising either a 5′-restriction pair member or a 3′-restriction pair member and a second cassette comprising both a 5′-restriction pair member and a 3′-restriction pair member and further comprising:

1) providing the first cassette with a first flanking restriction site at one end, either 5′ or 3′, of its insert sequence;

2) providing the second cassette with a second flanking restriction site that is, or is made, ligation compatible with the first flanking site and is on the same side, either 5′ or 3′, of its insert sequence as the first flanking restriction site is relative to the first cassette's insert sequence;

3) digesting the first cassette at its restriction pair member and the first flanking site and isolating the first fragment containing the insert sequence;

4) digesting the second cassette at its restriction pair member partner to the first cassette's restriction pair member and at the second flanking site and isolating the second fragment containing the insert sequence;

5) ligating the first fragment with the second fragment to generate a multimer cassette.

The identities of the first and second cassettes can vary. For example, the first cassette can be a 3′-terminal cassette and the second cassette an amplification cassette, the first cassette can be a 5′ terminal cassette and the second cassette an amplification cassette, the first cassette can be a 3′-terminal cassette and the second cassette a multimer 20 cassette constructed from a 5′-terminal cassette and an amplification cassette, or the first cassette can be a 5′-terminal cassette and the second cassette a multimer cassette constructed from a 3′-terminal cassette and an amplification cassette.

For the case when the first cassette is a 3′-terminal cassette and the second cassette is an amplification cassette, if the amplification cassette is digested at its 3′ restriction pair member and a flanking restriction site on the 5′ side of its 5′ restriction member to generate a ligatable fragment, then the 3′-terminal cassette is digested at its 5′ restriction pair member and a flanking restriction site on the 5′ side of this member to generate a ligatable cassette. Alternatively, if the amplification cassette is digested at its 3′ restriction pair member and a flanking restriction site on the 3′ side of this member to generate a ligatable cassette, then the 3′-terminal cassette is digested at its 5′ restriction pair member and a flanking restriction site on the 3′ side of its complete insert to generate a ligatable fragment.

It is important to note that the ligation of cassettes together does not require flanking sites. However, flanking sites enable oriented ligations. For example, if flanking sites are absent, a method of making a multimer cassette from two cassettes from a multimer assembly comprising a first cassette comprising either a 5′-restriction pair member or a 3′-restriction pair member and a second cassette comprising both a 5′-restriction pair member and a 3′-restriction pair member comprises:

1) digesting the first cassette at its restriction pair member and isolating the first fragment containing the insert sequence;

2) digesting the second cassette at both its restriction pair member sites and isolating the second fragment containing the insert sequence;

3) ligating the first fragment with the second fragment and screening for correct ligation orientation to generate a multimer cassette.

Again, the identities of the first and second cassettes can vary. The first cassette can be a 3′-terminal cassette and the second cassette an amplification cassette, the first cassette can be a 5′-terminal cassette and the second cassette an amplification cassette, the first cassette can be a 3′-terminal cassette and the second cassette a multimer cassette constructed from a 5′-terminal cassette and an amplification cassette, or the first cassette can be a 5′-terminal cassette and the second cassette a multimer cassette constructed from a 3′-terminal cassette and an amplification cassette.

FIG. 5 illustrates a method of making an insertion cassette from the 5′-terminal cassette and the 3′-terminal cassette when each shares an insertion restriction site. The method comprises:

1) providing the 5′-terminal cassette with a first flanking restriction site, independent of the insertion restriction site, that is outside of the sequence including the insert sequence and insertion restriction site of the 5′-terminal cassette;

2) providing the 3′-terminal cassette with a second flanking restriction site, independent of the insertion restriction site, that is outside of the sequence including the insert sequence and insertion restriction site of the 3′-terminal cassette and is, or is made, ligation compatible with the first flanking site and is on the same side, either 5′ or 3′, of its insert sequence as the first flanking restriction site is relative to the 5′-terminal cassette's insert sequence;

3) digesting the 5′-terminal cassette at its insertion restriction site and the first flanking site and isolating the first fragment containing the insert sequence;

4) digesting the 3′-terminal cassette at its insertion restriction site and the second flanking site and isolating the second fragment containing the insert sequence;

5) ligating the first fragment with the second fragment to generate an insertion cassette.

FIG. 6 illustrates a method of making a multimer cassette comprising an insertion cassette and an amplification cassette from a multimer assembly comprising:

1) digesting the insertion cassette at both its restriction pair member sites and isolating the first fragment containing the insert sequence;

2) digesting the amplification cassette at both its restriction pair member sites and isolating the second fragment containing the insert sequence;

3) ligating the first fragment with the second fragment and screening for correct ligation orientation to generate a multimer cassette.

FIGS. 7 and 8 illustrate a method of making a multimer cassette comprising an insertion cassette and an amplification cassette comprising:

1) digesting the amplification cassette at the insertion restriction site and its restriction pair member on the opposite side, either 5′ or 3′, of the insert sequence and isolating the first fragment containing the insert sequence;

2) digesting the insertion cassette at the insertion restriction site and the restriction pair member partner to the digested amplification cassette's restriction pair member and isolating the second fragment containing the insert sequence;

3) ligating the first fragment with the second fragment to generate a multimer cassette precursor;

4) digesting the multimer cassette precursor at both restriction pair members, isolating the fragment containing the insert sequence, and ligating it with itself to generate a multimer cassette.

Once constructed, the gene for the multimer can be used as an insert to construct other cassettes or to express it in a suitable transcription and translation system. Once isolated in the correct conformation and with the necessary degree of purity, polymeric polypeptides are available for applications in the fields of medicine, veterinary care, research and development, diagnostics, etc. The present invention comprises proteins made from multimer assemblies of the present invention.

Each cassette can involve a fusion of any of a number of functional elements. For example, any construction involving a linker is by nature a heteromultimer, because the monomer contains at least two functional elements. A particularly expeditious method to produce these fusions is to treat each functional element as a nested assembly. In other words, each element itself is an assembly that consists of individual cassettes.

The current methods are easily extended to heteromultimers if two sequences share compatible restriction sites. For instance, two distinct monomer amplification cassettes, A and B, can be ligated together if they share the same restriction pair. Subsequent polymerization of this new “monomer” results in an alternating sequence, ABAB . . . . Any pattern of alternating sequences can theoretically be constructed from any number of initial monomers. For example, the pattern ABBCABBC . . . is just one possibility.

### II Multimer Assemblies and Multimer Cassettes

The present invention includes multimer assemblies made using the methods of the present invention and novel cassettes incorporating novel restriction pair members. In some preferred aspects of the present invention, a multimer assembly of the present invention comprises two or more amplification cassettes, in which fused 5′ and 3′ restriction pair member sites join the amplification cassettes. An amplification cassette can comprise any practical number of monomer sequences.

Multimer assemblies of the present invention comprise component constructs having 5′ restriction pair members, 3′ restriction pair members, or both 5′ restriction pair members and 3′ restriction pair members that can be used to make multimer cassettes, including multimer expression cassettes. Such cassettes are synthesized by joining component cassettes (such as 5′-terminal cassettes, 3′-terminal cassettes, and amplification cassettes) by ligating a 3′ restriction pair member site of one component cassette to a 5′ restriction pair member site of another component cassette.

One multimer assembly of the present invention comprises one or more amplification cassettes and at least one 3′-terminal cassette. Another multimer assembly of the present invention comprises one or more amplification cassettes and at least one 5′-terminal cassette. Another multimer assembly of the invention comprises one or more amplification cassettes, at least one 3′-terminal cassette, and at least one 5′-terminal cassette.

Multimer expression cassettes made from multimer assemblies of the present invention include, for example, multimer cassettes in which a 5′-terminal cassette is fused to an amplification cassette comprising a single monomer, multimer cassettes in which a 5′-terminal cassette is fused to a multimer amplification cassette constructed from multiple amplification cassettes, and multimer cassettes in which a 5′-terminal cassette is fused to a multimer cassette comprising one or more amplification cassettes and at least one 3′-terminal cassette. Multimer expression cassettes made from multimer assemblies of the present invention also include, for example, multimer cassettes in which a 3′-terminal cassette is fused to an amplification cassette, multimer cassettes in which a 3′-terminal cassette is fused to a multimer amplification cassette constructed from multiple amplification cassettes, and multimer cassettes in which a 3′-terminal cassette is fused to a multimer cassette comprising one or more amplification cassettes and at least one 5′-terminal cassette.

The present invention also includes novel amplification cassettes. In one aspect of the present invention, an amplification cassette comprises at least one linker, in which at least one of the one or more linkers comprises at least one restriction pair partner. Amplification cassettes can be fused using restriction pair partners, at least one of which is introduced in the linker, to form a multimer amplification cassette. The method of making the multimer amplification cassette is by joining two or more amplification cassettes by ligating the first restriction pair partner of at least one of the two or more amplification cassettes to the second restriction pair partner of at least one other of the two or more amplification cassettes to generate a multimer cassette. The present invention includes multimer amplification cassettes comprising component amplification cassettes that incorporate linkers, and multimer assemblies and multimer expression cassettes that include such multimer amplification cassettes.

Also included as amplification cassettes of the present invention are amplification cassettes that comprise monomer sequences in noncontiguous orientation. For example, an amplification cassette can comprise a 5′ segment of a monomer sequence and a 3′ segment of a monomer sequence that together comprise the sequence of a complete monomer, in which the 5′ segment is positioned 3′ of the 3′ monomer segment. In these embodiments, the 5′terminus of the 3′ monomer segment is preferably a 5′ restriction pair member and the 3′ terminus of the 5′ monomer segment is preferably a 3′ restriction pair member. The present invention also includes multimer amplification cassettes comprising two or more amplification cassettes that comprise monomer sequence in noncontiguous orientation. Such multimer cassettes comprising multiple amplification cassettes can be made by ligating a 3′ restriction member of at least one of the two or more amplification cassettes to a 5′ restriction member of at least one other of the two or more amplification cassettes. The present invention also includes multimer assemblies and multimer expression cassettes that include such amplification and multimer amplification cassettes.

In yet another aspect, the present invention includes amplification cassettes that comprise 3′ and 5′ restriction pair members comprising restriction sites that are initially ligation incompatible but are blunt ended to make them ligation compatible. The present invention also includes multimer amplification cassettes comprising two or more amplification cassettes that comprise noncompatible sites that have been blunt-ended and then ligated to join the two or more amplification cassettes. The present invention also includes multimer assemblies and multimer expression cassettes that include such amplification and multimer amplification cassettes.

The invention includes multimer assembly cassettes in vectors, including cloning and expression vectors, where expression vectors can be designed for in vitro or in vivo expression. The vectors can be designed for in vivo expression in prokaryotes or eukaryotes, including but not limited to, bacterial cells, fungal cells, algal cells, plant cells, insect cells, avian cells, and mammalian cells. The present invention also encompasses cells that include such vectors and polymeric proteins made using vectors that comprise multimeric expression vectors of the present invention. The present invention also encompasses polymeric proteins expressed from the multimeric assemblies of the present invention.

The disclosed invention also encompasses the construction of different multimer assemblies involving multimeric hGH, and multimer cassettes made using the methods of the present invention that comprise multimerized hGH sequences or multimerized portions of hGH. Sequences encoding hGH or portions thereof that are part of multimer cassettes and multimer assemblies of the present invention include sequences that encode hGH taking into account the redundancy of the genetic code. Sequences encoding hGH or portions thereof that are part of multimer cassettes and multimer assemblies of the present invention include sequences that encode hGH can also comprise sequence changes with respect to the human GH gene sequence that change the amino acid sequence where such changes do not detrimentally affect the activity of the protein or portion thereof.

The hGH assemblies can differ in the functional elements included, such as those provided by 3′- or 5′-terminal elements. The ease of producing these assemblies, and the resulting multimers and polymers, demonstrates the utility of the methods disclosed. In the examples below, restriction sites outside, and flanking, the restriction pair sites are engineered in order to facilitate the manipulation of the cassettes.

Endogenous hGH appears in several forms in vivo as a result of expression from more than one gene, as well as alternative gene splicing. The predominant mature form of hGH is a single polypeptide chain consisting of 191 amino acids. The DNA and protein sequences for this predominant form are given as SEQ ID NO: 1 and SEQ ID NO: 2, respectively.

In the following paragraphs, the term “engineer” refers to using standard techniques of molecular biology generally known to those skilled in the art. Standard techniques include, but are not restricted to, restriction digestion and ligation, PCR amplification and mutagenesis, DNA synthesis, DNA isolation and purification, etc., as described in Sambrook et al. (2000), which are hereby incorporated by reference. As such, the details are only described if they bear directly on the present invention or deviate from common practice.

### EXAMPLES

A drawback to rhGH therapy is the need for once daily injections. Understandably, patient preference is for a minimum of injections. In an attempt to overcome this, rhGH has been formulated with PLGA in microspheres, chemically linked to PEG, and fused to HSA in order to produce longer acting versions. Here we describe the construction of families of multimeric rhGHs, according to the steps below using the general procedures shown in FIGS. 1 to 8.

### Example 1

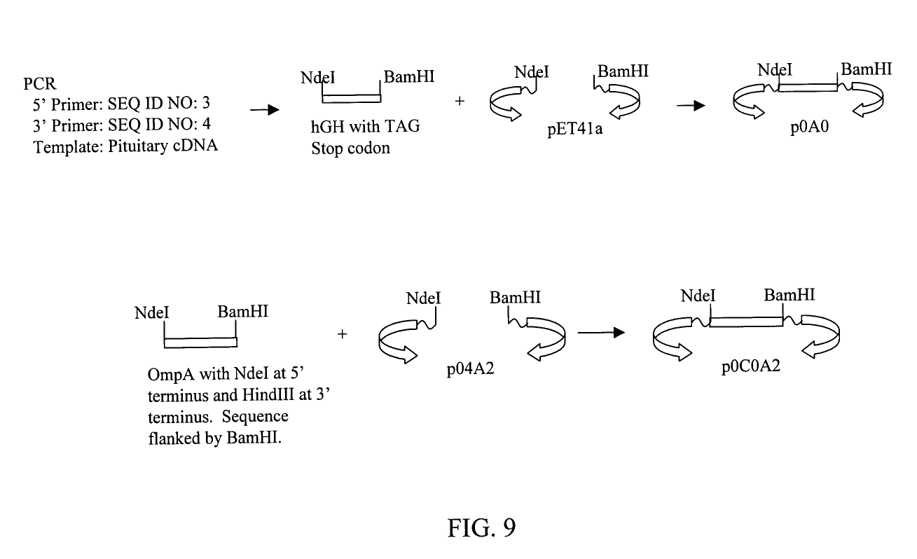

The first example involves isolation of the GH gene. Steps to isolate the hGH gene are summarized in FIG. 9. hGH is highly expressed in the anterior pituitary gland. As a result, mRNA of hGH is abundantly found in lysates of human pituitary. The gene for hGH is PCR amplified from human pituitary cDNA (Human Pituitary Gland Quick-CloneTM cDNA, BD Biosciences Clontech, Palo Alto, Calif., catalog #7173-1) using SEQ ID NO: 3 as the 5′ primer and SEQ ID NO: 4 as the 3′ primer. The 5′ primer has an NdeI restriction enzyme site coding for an N-terminal methionine, and the 3′ primer has a BamHI restriction enzyme site immediately after the TAG stop codon. The resulting PCR fragment is isolated from the reaction mix using standard techniques, as are all subsequent ones.

The purified PCR fragment is ligated into parent plasmid pET41a (Novagen, Madison, Wis.) after both insert and plasmid are digested with NdeI and BamHI and purified, again using standard techniques. This plasmid ligation mixture, and all others unless otherwise indicated, is transformed into DH5α cells and plated on LB/antibiotic plates. Single colonies are sub-cultured and plasmid DNA is isolated from each. Restriction enzyme analysis is used to confirm the presence of an insert into the plasmid, and plasmids with insert are sent for DNA sequencing using SEQ ID NO: 5 and SEQ ID NO: 6 (Novagen, Madison, Wis.) as amplification primers for the 5′ and 3′ ends, respectively. Plasmid with correct insert is identified as p0A0, and the DNA coding region and corresponding open reading frame (ORF) translation are listed in SEQ ID NO: 7 and SEQ ID NO: 8, respectively. The convention for the sequences is that the restriction sites are included at the termini of DNA sequences and only translated amino acids that eventually appear in an expressed insert are given. Expression of protein from p0A0 yields a 192 amino acid protein consisting of full length hGH with an additional N-terminal methionine.

It is convenient to engineer a high copy number plasmid that contains the hGH gene and enables digestion of the hGH gene in its interior so that 5′ or 3′ elements can be swapped in and out. The gene for hGH contains a convenient PstI site, CTGCAG. The plasmid p04 (SEQ ID NO: 9), a derivative of pUC19 (New England Biolabs) containing the same multi-cloning site as pET41a, is first readied by digesting with PstI, followed by Mung Bean Nuclease, and subsequent re-ligation to destroy the internal PstI site to create p04A1. Finally, the NdeI/BamHI hGH fragment from p0A0 is ligated into similarly digested p04A1 to yield p0A03.

Several examples are now given to generate assemblies for GH multimers with different linkers. Variation in the linker sequence, as well as the degree of monomer polymerization, may alter the polymers ease of production, conformation, in vitro activity, in vivo activity, immunogenicity, etc.

### Example 2

The second example involves generation of an assembly for the direct fusion multimer of GH.

There is not a convenient restriction pair at the termini of rhGH, so this example uses the methods for a monomer sequence with an internal restriction pair. A direct fusion assembly for hGH is constructed with the features diagrammed in FIG. 2. Disclosed are two 5′-terminal cassettes, the amplification cassettes, and two 3′-terminal cassettes. The 3′-terminal cassette is engineered to enable construction of an insertion cassette, as shown in FIG. 5. This facilitates insertion of amplification cassettes to generate expressible genes for different size homopolymeric GHs.

Two 5′-terminal cassettes for the GH fusion protein assembly are disclosed. The first is a direct start 5′-terminal cassette, and the second is an OmpA start 5′-terminal cassette. The direct start results in an N-terminal methionine at the N-terminus of the final expressed GH polymer. Its construction is straight forward because the insert in p0A0 and p0A03 already has the N-terminal methionine fused to the GH gene. In contrast, the OmpA start codes for an N-terminal leader sequence that targets the polymer to the periplasmic space of E. Coli, resulting in the cleavage of the leader from the polymer. There are many other 5′-terminal cassettes that can easily be generated by those skilled in the art.

A pre-5′-terminal cassette is disclosed that enables fusion of the OmpA sequence to any other blunt end or HindIII digested sequence. SEQ ID NO: 10 is a synthetic DNA fragment that contains the coding sequence for the OmpA leader peptide, and its ORF translation is listed in SEQ ID NO: 11. The fragment has a 5′ NdeI site, the OmpA leader coding region, a 3′ HindIII site for HindIII ligation or blunt end ligation after filling in the HindIII 5′ overhang with T4 DNA polymerase, and a BamHI site for cloning flexibility. Plasmid p04 is readied by digestion to destroy an internal site, this time the HindIII site. The plasmid is digested with HindIII, followed by Mung Bean Nuclease, and subsequently ligated back together to create p04A2. Both p04A2 and insert DNA are digested with NdeI and BamHI and ligated together to yield the plasmid p0C0A2 as shown in FIG. 9.

For the current use, a GH sequence is needed that contains a 5′ blunt end or HindIII site, along with a 3′ restriction site that is the 3′ member of a restriction pair. The 5′ terminus is engineered with a HindIII site. Digestion with Mung Bean Nuclease after digestion with HindIII results in a blunt 5′ end that leaves the 5′-terminal codon of GH, TTC, intact. Although the blunt end is not needed for the current example, in general it is necessary for ligation to other hypothetical cassettes.

There are several choices for the restriction site pair, and we choose to use GH amino acids 187 and 188, glycine and serine, that are compatible with, among other enzymes, BamHI and BclI. The two enzymes recognize sequences GGATCC and TGATCA, respectively. BamHI is assigned as the 3′ member, and BclI is assigned as the 5′ member.

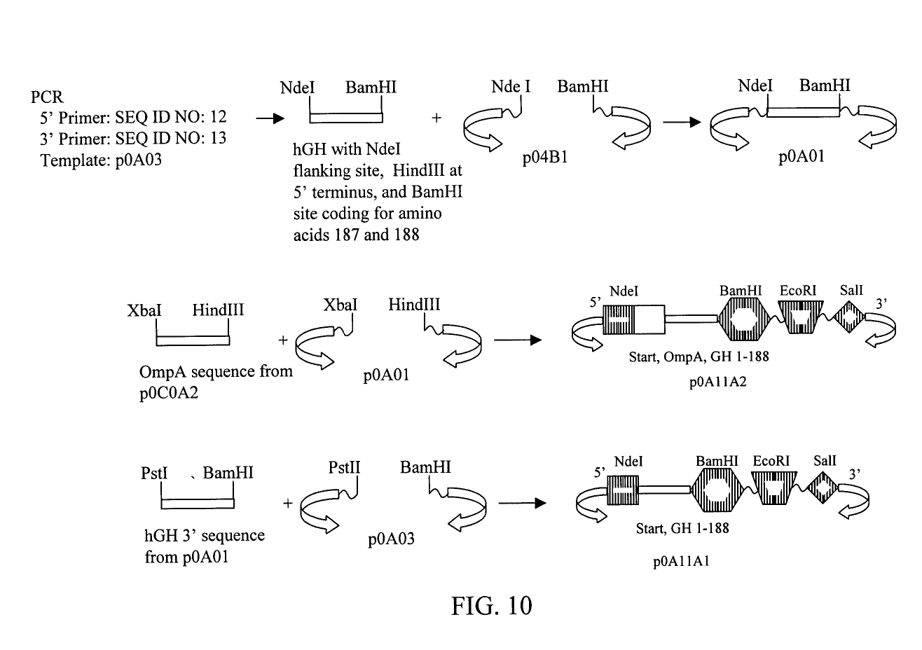

The desired DNA sequence is generated by PCR using p0A03 as template, as shown in FIG. 10. The 5′ and 3′ primers are listed in SEQ ID NO: 12 and SEQ ID NO: 13, respectively, and the DNA coding region for the insert between the 5′ flanking NdeI and 3′ BamHI sites is listed in SEQ ID NO: 14. The fragment is digested with HindIII and BamHI and inserted into similarly digested p04B1 to yield p0A01. Plasmid p04B1 is prepared by destroying the HindIII site in p04A1 as described for the preparation of p04A2. The result is a parent plasmid with the PstI and HindIII sites destroyed.

The 5′-terminal cassettes are now constructed from the generated sequences as shown in FIG. 10. The XbaI/HindIII fragment from p0C0A2 is inserted into plasmid p0A01 to generate p0A11A2. The result is the OmpA 5′-terminal cassette for the GH direct fusion assembly. It contains the OmpA sequence fused directly to the 5′ coding region of GH. The resulting DNA insert between NdeI and BamHI is listed in SEQ ID NO: 15, with corresponding ORF listed in SEQ ID NO: 16. The direct translation start 5′-terminal cassette is constructed by ligating fragments from existing sequences. The PstI/BamHI 5′ GH fragment and plasmid backbone that results from digesting p0A03 is ligated with the PstI/BamHI 3′ GH fragment that results from digesting p0A01 to yield p0A11A1. The resulting DNA sequence between NdeI and BamHI, and the corresponding ORF, for p0A11A1 are listed in SEQ ID NO: 17 and SEQ ID NO: 18, respectively.

As shown in FIG. 2, the amplification cassette must contain several components. First, it must have both the 5′ and 3′ members of the restriction pair to enable polymerization. In between must be the entire continuous GH sequence. Finally, if convenient, there should be flanking restriction sites for insertion and extraction of the sequence from a plasmid backbone.

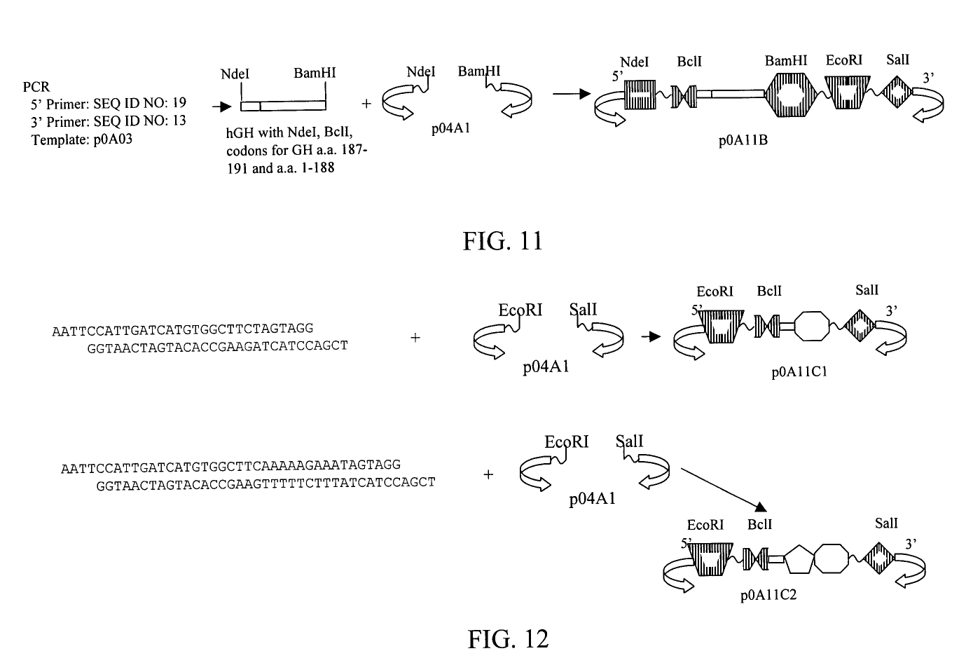

The amplification cassette for the current direct fusion of GH is generated by PCR, as shown in FIG. 11. The 5′ primer is listed in SEQ ID NO: 19. It contains an NdeI site, the 5′ restriction pair member BclI, followed by the codons that together code for GH amino acids 187-191, and finally codons to anneal to the GH 5′-terminal codons. The 3′ primer is one previously used and listed in SEQ ID NO: 13. The PCR template is p0A03. The resulting insert DNA sequence between NdeI and BamHI is listed in SEQ ID NO: 20, with ORF sequence listed in SEQ ID NO: 21. The DNA sequence is inserted into plasmid p04A1 to yield p0A11B.

Two simple 3′-terminal cassettes are disclosed, as shown in FIG. 12. Both code for the 3′ terminus of GH, starting at the glycine and serine codons within the BclI site, amino acids 187 and 188, and ending with the translation stop codon, TAG. The first cassette, given in SEQ ID NO. 22, is a direct translation stop. The double stranded DNA is synthesized and contains an EcoRI site flanking the 5′ terminus, a BclI site to ligate to BamHI, the 3′ terminus of GH, a stop codon, and a SalI site for cloning flexibility. It is inserted into p04A1 by digesting the synthetic DNA and p04A1 with EcoRI and SalI and ligating the large fragments together to yield plasmid p0A11C1. The C-terminal ORF protein sequence contributed by this cassette to subsequent GH multimer constructs is given in SEQ ID NO: 23.

The second 3′-terminal cassette, given in SEQ ID NO: 24, is a synthetic DNA fragment similar to the first, except it contains the codons for a 3 amino acid polylysine tail before the stop codon. It is analogously inserted into p04A1 to yield plasmid p0A11C2. The polylysine tail is potentially useful for chemical conjugation with other molecules. SEQ ID NO: 25 is the C-terminal ORF sequence contributed by the new insert to subsequent GH multimer constructs.

Once the basic cassettes are complete, the amplification cassette can be polymerized, the 5′-terminal and 3′-terminal cassettes can be joined to form an insertion cassette, and finally amplification cassettes can be ligated to the insertion cassette to generate expressible multimers.

### Example 3

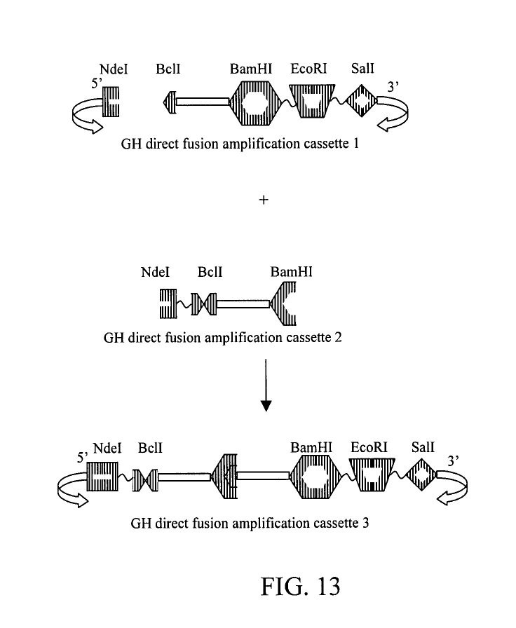

The polymerization of the GH direct fusion amplification cassettes is performed as shown in general in FIG. 3 and specifically in FIG. 13. The first polymerization is formation of the dimer. Plasmid p0A11B is digested with NdeI and BclI and the plasmid isolated. In a separate reaction, p0A11B is digested with NdeI/BamHI and the insert isolated. The two fragments are then ligated together to yield plasmid p0A11B2. Its insert DNA sequence is listed in SEQ ID NO: 26, and the corresponding ORF translation is listed in SEQ ID NO: 27. This process is repeated, changing the identity, and thus the size, of amplification cassettes 1 and 2 in FIG. 13 to construct polymer inserts of different sizes. The size of new constructs is increased fastest if the polymerization is done geometrically, each time using the most recent construct for both cassettes 1 and 2. The size is increased by one if the monomer amplification cassette, p0A11B, is used either as cassette 1 or 2. The generalized sequences for the resulting amplification cassettes are given in SEQ ID NO: 28 and SEQ ID NO: 29 for the DNA and protein, respectively.

### Example 4

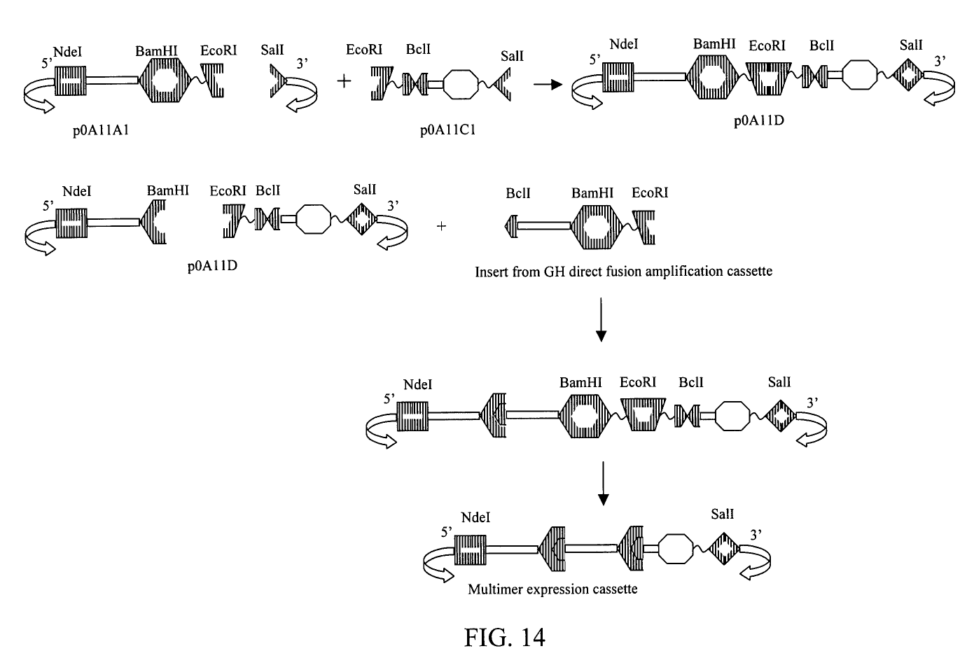

The cassettes for the GH direct fusion assembly are designed to enable construction of insertion cassettes to facilitate generation of a variety of expressible polymers. The general procedures are shown in FIGS. 5 and 7 and the specifics in FIG. 14. Different insertion cassettes can be generated with the various 5′-terminal and 3′-terminal cassettes. However, only the one involving p0A11A1 and p0A11C1 is described here. Others are constructed in exactly the same way.

Plasmid p0A11A1 is digested with EcoRI and SalI and the opened plasmid is isolated. Plasmid p0A11C1 is digested with the same enzyme pair and the insert isolated. The two fragments are ligated together to generate the insertion cassette, p0A11D, and the resulting DNA sequence is listed in SEQ ID NO: 30. Plasmid p0A11D is compatible with ligation of any of the amplification cassettes for this assembly. It need be prepared only once for all subsequent ligations, as long as the supply is sufficient.

### Example 5

Either of the two schemes shown in FIGS. 6 and 7 can be used to ligate amplification cassettes into the insertion cassette. The example given here utilizes the oriented ligation shown in FIG. 7 and subsequent digestion and re-ligation to generate final products as shown in FIG. 14.

Plasmid p0A11D is digested with BamHI and EcoRI, and the plasmid is isolated. An amplification cassette is digested with BclI and EcoRI and the insert isolated. Ligation of the two fragments yields an intermediate that is converted to the multimer expression cassette after digestion with BamHI and BclI, purification, and subsequent re-ligation. The result is an expression ready insert for the direct fusion growth hormone multimer. When performed with the Nmer amplification cassette, the result is an N+1 multimer expression cassette. The insert has general DNA sequence listed in SEQ ID NO: 31 and corresponding ORF translation listed in SEQ ID NO: 32. The production of different size multimers is controlled by the size of the ligated amplification cassette.

Protein expression is achieved by digesting and ligating the multimer expression cassette insert into an appropriate expression system. For example, the insert can be liberated with NdeI and SalI and ligated into similarly digested pET41a, followed by transformation into E. coli strain BL21(DE3) (Novagen).

One utility of the invention is the ease of production of different size multimers and different variations once the basic cassettes, p0A11A1, p0A11A2, p0A11B, p0A11C1, and p0A11C2, for example, are constructed. Those skilled in the art can easily see how substituting p0A11C2 for p0A11C1 when generating the insertion cassette generates a polylysine tail variant.

### Example 6

The next example involves generation of a GH multimer with a linker without a convenient restriction pair. The one amino acid linker, glycine, is used as an example. The construction of GH multimers with a glycine linker is analogous to the construction of the fusion protein. In fact, the GH glycine linker assembly shares the same 5′- and 3′-terminal cassettes with the GH fusion protein assembly. This is one advantage of the assembly construction scheme given in FIG. 2. Assemblies differing only in the linker region only need different amplification cassettes, while sharing the same 5′- and 3′-terminal cassettes.

Use p0A11A1 and p0A11A2 as before for the direct start and OmpA 5′-terminal cassettes for the direct fusion assembly. Use p0A11C1 and p0A11C2 as before for direct stop and poly lysine 3′-terminal cassettes.

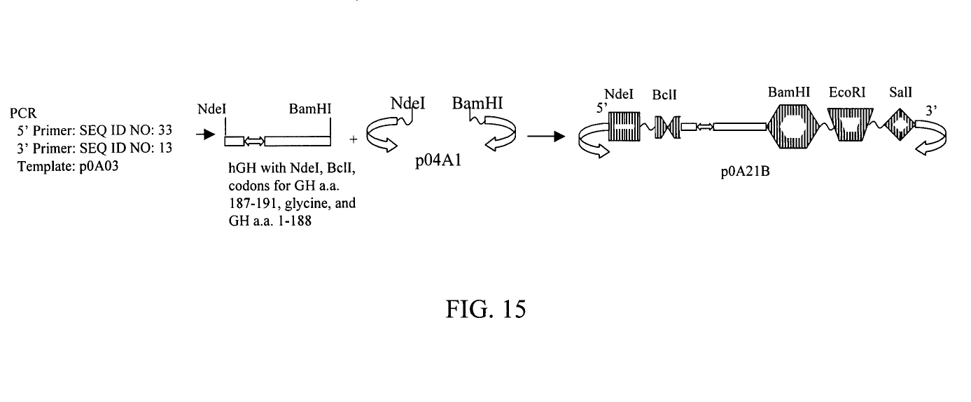

The only difference is the amplification cassettes that contain a glycine codon between the ending and starting codons for GH. The glycine linker amplification cassette is made in the same way as the one for the direct fusion homomultimer except for some necessary substitutions of sequences, as shown in FIG. 15. SEQ ID NO: 33 is substituted for SEQ ID NO: 19 as the 5′ PCR primer. It contains the same elements as before, as well as the glycine codon between the sequence for amino acids 191 and 1. The resulting PCR fragment is inserted into parent plasmid p04A1 by digesting both the parent plasmid and the PCR fragment with NdeI and BamHI and ligating the appropriate fragments together. The resulting plasmid is labeled p0A21B. The DNA sequence and ORF translation for the insert sequence between NdeI and BamHI are listed in SEQ ID NO: 34 and SEQ ID NO: 35, respectively.

The construction of additional amplification assemblies, the insertion cassette, and multimer expression cassettes for the GH glycine linker assembly is identical in practice to the one for the GH direct fusion assembly, FIGS. 13 and 14, except for the substitution of p0A21B for p0A11B. The corresponding generalized amplification cassette insert DNA and ORF sequences are listed in SEQ ID NO: 36 and SEQ ID NO: 37, and the general formulas for the multimer expression cassettes are listed in SEQ ID NO: 38 and SEQ ID NO: 39.

The previous examples have demonstrated, among other things, the ease at which multiple 5′- and 3′-terminal cassettes can be used to introduce variations in the N- and C-termini of a polymer. In the case of the 5′-terminal cassettes, cassettes with either a direct translation start or one introducing a leader sequence are disclosed. In the case of the 3′-terminal cassettes, ones with either a direct stop or one introducing a polylysine tail are disclosed. Each demonstrates the ease at which functional elements can be added to the beginning or end of a polymer sequence. These methods are easily extended to other examples by those skilled in the art. Therefore, subsequent examples will be limited to the presentation of only a single 5′- and 3′-terminal cassette for each assembly.

The next examples involve generation of GH multimers utilizing linkers that result in monomers with a terminal restriction pair. FIG. 1 details the general features for these assemblies.

### Example 7

This example involves a linker that is noteworthy because it contains a 3′ restriction pair member with a functional stop codon that is destroyed upon polymerization. Use of this linker makes it possible to express functional multimers using just the 5′-terminal and amplification cassettes. However, a 3′-terminal cassette is necessary to express homomultimers without any residual linker at the 3′ terminus of the protein.

The 5′ restriction pair member is NcoI, C\hat{ }CATGG, while the 3′ restriction pair member is RcaI, T\hat{ }CATGA. Therefore, the resulting linker sequence is A-Ser-Trp-B, where A and B are arbitrary protein sequences. For the given example, A is a null sequence, and B is G₄S, where the single letter amino acid abbreviations are used.

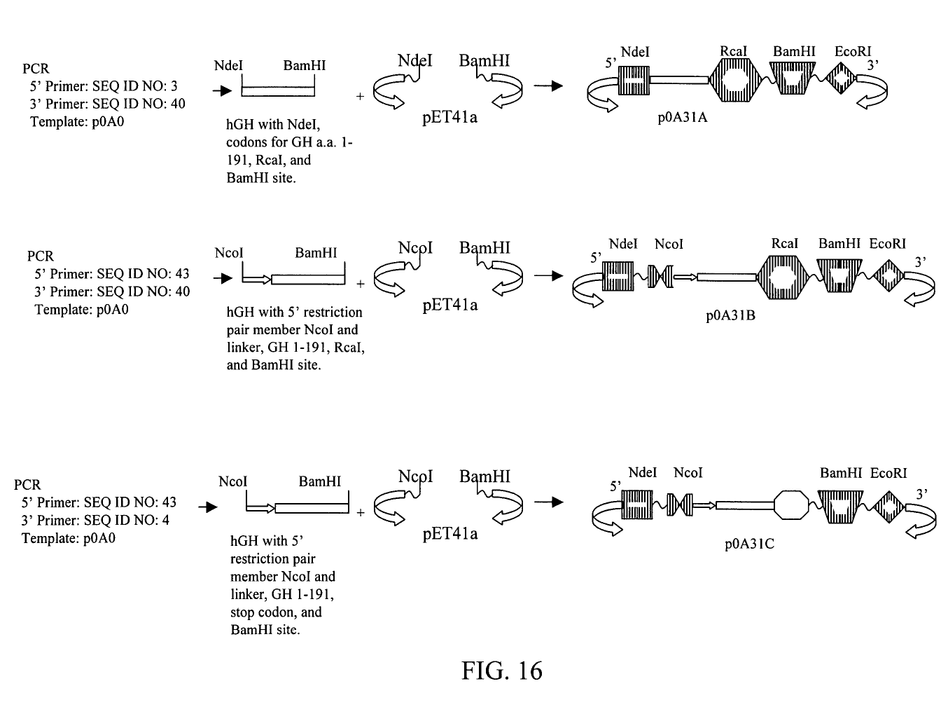

For this example, only one 5′-terminal cassette is disclosed, with a direct ATG start codon and no leader sequence, as shown in FIG. 16. The PCR primers for the 5′-terminal cassette are listed in SEQ ID NO: 3 and SEQ ID NO: 40, for the 5′ and 3′ ends, respectively. The 5′ primer maintains the NdeI site and its start codon, while the 3′ primer introduces a stop codon within an RcaI (or BspHI) restriction site, immediately followed by a BamHI site. The template for the reaction is p0A0.

Because the RcaI restriction site also contains the codon TCA immediately 5′ of the stop codon, it also introduces a C-terminal serine residue. The resulting PCR fragment is purified and ligated into pET41a in an analogous manner for the generation of p0A0. The sequence verified plasmid is labeled p0A31A, and the DNA coding region, from the NdeI to the BamHI site, and the resulting ORF protein sequence are listed in SEQ ID NO: 41 and SEQ ID NO: 42, respectively. Expression of protein from the gene for p0A31A yields a 193 amino acid protein consisting of full length hGH with an additional N-terminal methionine and C-terminal serine.

The PCR primers for the amplification cassette are listed in SEQ ID NO: 43 and SEQ ID NO: 40, for the 5′ and 3′ ends, respectively. The 5′ primer introduces an NcoI site followed by the linker region. The NcoI site is ligation compatible with the 3′ RcaI site, and any such ligation destroys the TGA stop codon by altering it to a TGG codon. The resulting PCR fragment is purified and ligated into pET41a after the PCR product and plasmid are cut with NcoI and BamHI, as shown in FIG. 16. The sequence verified plasmid is labeled p0A31B, and the DNA coding region from the NcoI to the BamHI site is listed in SEQ ID NO: 44. The ORF protein sequence coded by the insert is given in SEQ ID NO: 45.

Again, for this example, only one 3′-terminal cassette is disclosed, with a direct TAG-stop codon and no other 3′-specific sequences. The 3′-terminal cassette is constructed using PCR with p0A0 as template and SEQ ID NO: 43 and SEQ ID NO: 4 as 5′ and 3′ primers, respectively. This creates a cassette with a 5′ linker and a 3′ stop codon immediately following the last amino acid from the parent monomer. The PCR fragment is inserted into pET41a as before and shown in FIG. 16 to create p0A31C. The resulting DNA and protein fragments between the NdeI and BamHI sites are listed in SEQ ID NO: 46 and SEQ ID NO: 47, respectively.

The scheme for the polymerization of the amplification cassettes is shown in FIG. 3. Additional care is necessary because the parent plasmid contains RcaI sites. One way to unambiguously liberate the insert sequence for polymerization is to first digest the flanking BamHI site, isolate the insert, and then digest with RcaI. The general formulas for the Nmer amplification cassette are listed in SEQ ID NO: 48 and SEQ ID NO: 49 for the DNA and corresponding ORF translation, respectively.

### Example 8

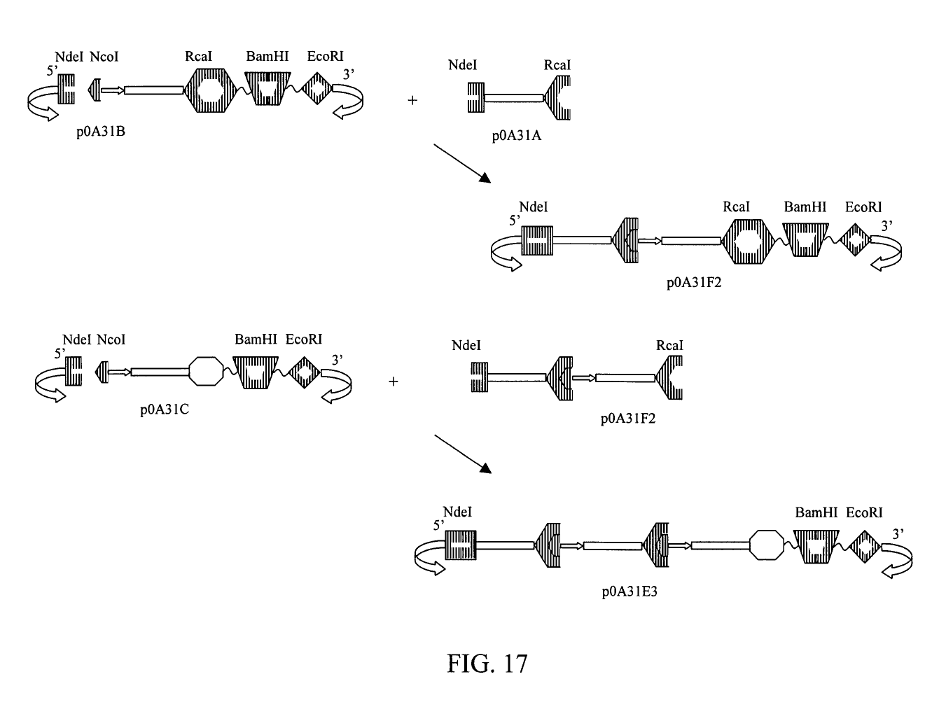

The ligation of the multimer assembly cassettes must be done sequentially, as shown in FIG. 4, because the arrangement of the restriction sites in the 3′-terminal cassette is like FIG. 2d. The first ligation involves the 5′-terminal and amplification cassettes, rather than the 3′-terminal and amplification cassettes, to take advantage of the stop codon in the 3′-restriction member to produce expression ready inserts. The specifics are shown in FIG. 17 using procedures already described. Use of the monomeric amplification cassette, p0A31B, results in the dimeric cassette, p0A31F2, with insert DNA and corresponding ORF translation listed in SEQ ID NO: 50 and SEQ ID NO: 51. The general formulas for the N+1 mer produced after ligation between the Nmer amplification and the 5′-terminal cassettes are listed in SEQ ID NO: 52 and SEQ ID NO: 53. Transfer of the insert into an appropriate expression system yields expression of the N+1 GH polymer with the SWG4S linker and C-terminal S residue.

Completion of the ligation scheme shown in FIG. 17 results in an insert with an additional monomer and the natural C-terminus of GH. If the insert from p0A31F2 is ligated into p0A31C, then the trimer expression cassette p0A31E3 is generated. In general, the formulas for the insert DNA and corresponding ORF translation when the Nmer amplification cassette is used are listed in SEQ ID NO: 54 and SEQ ID NO: 55. For p0A31E3, the monomer amplification cassette is used and N=1.

### Example 9

The plasmids containing the inserts generated with the ligation scheme shown in FIG. 17 are capable of expressing rhGH polymers following standard techniques (see for example, user manuals from Novagen, Madison, Wis.). DNA sequences listed in SEQ ID NO: 52 with N=0, 1, 2, 4, and 8 and prepared according to Example 8 are ligated into pET41a. The resulting plasmids are separately transformed into BL21(DE3) and separately grown in Luria Browth medium and induced to express the polymer protein by adding IPTG to a concentration of 1 mM.

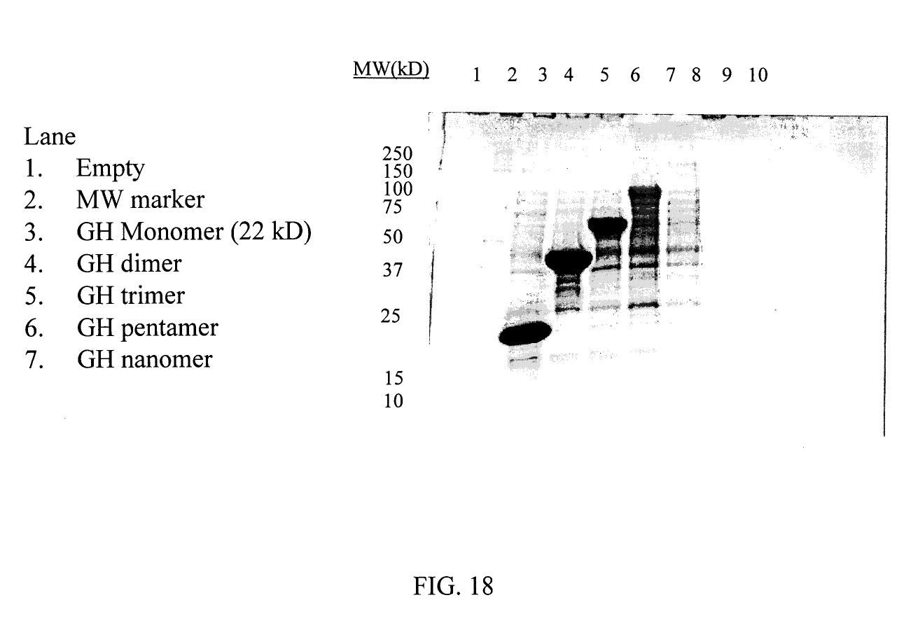

Following 3 hours of induction, each culture is harvested by centrifugation and treated with SDS-PAGE sample buffer. Proteins from the samples for each culture are separated according to their molecular weights on a standard SDS-PAGE gel (Invitrogen, Carlsbad, Calif.). The resulting gel is stained with coomasie blue stain to visualize the protein bands Results for the monomer (SEQ ID NO: 42), dimer (N=1 in SEQ ID NO: 53), trimer (N=2 in SEQ ID NO: 53), pentamer (N=4 in SEQ ID NO: 53), and nanamer (N=8 in SEQ ID NO: 53) are given in FIG. 18. As the figure demonstrates, large amounts of each polymeric rhGH are produced except for the nanamer.

### Example 10

Linkers with convenient restriction sites offer the engineering option to generate a multitude of assemblies with cassettes that can be attached to monomers using restriction/ligation techniques. The utility of this formulation lies in the breadth of assemblies that can be constructed relatively easily. This is especially apparent when the linkers themselves are treated as assemblies nested within the construction of the multimers. Once constructed, these linker assemblies and cassettes, like any other, can be reused to produce new assemblies.

Nested linker assemblies are constructed having a slightly different function than the multimer assemblies. They still need an amplification cassette for the polymerization of the linker. However, the other cassettes in the assembly enable attachment of the linker to either a 5′or 3′ terminus, whichever is appropriate.

The example given here is a series of linkers, having amino acid sequence GZGS, where Z is an arbitrary sequence of arbitrary length. The series of linkers in Table 1 below share features that enable them to be treated similarly in terms of their engineering. All but one has a Glycine at the N-terminus of the linker that can be coded by an NaeI restriction site at the 5′ end for blunt end ligation of a 5′-terminal cassette to a monomer pre-cassette. For the other linker, GS, a synthetic DNA fragment must be ligated to the monomer pre-cassette without propagation within a plasmid. Each of the linkers ends in the protein sequence GS, so that the restriction pair is identical to earlier examples utilizing the BclI and BamHI sites.

TABLE 1

| | | |
|---|---|---|
| Linker protein | 5′-terminal cassette DNA | Amplification cassette DNA |
| monomer unit | sequence | sequence |

| | | |
|---|---|---|
| GS | GGATCC | TGATCAGGATCC |
| GGS | GCCGGCGGATCC | TGATCAGGCGGATCC |
| GGGS | GCCGGCGGCGGATCC | TGATCAGGCGGCGGATCC |
| GGGGS | GCCGGCGGCGGCGGATCC | TGATCAGGCGGCGGCGGATCC |
| GZGS | GCCGGCYGGATCC | TGATCAGGCYGGATCC |
| | Z is an arbitrary protein | |
| | sequence, and Y is its DNA coding | |
| | sequence. | |

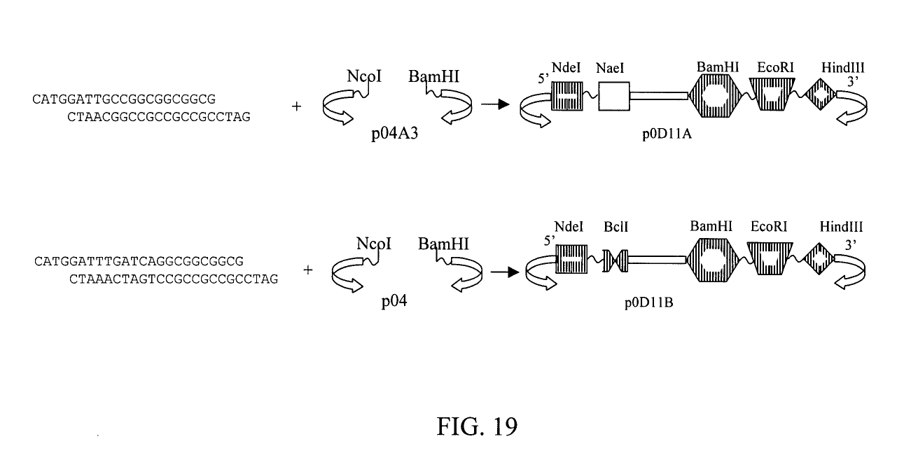

As a single example of the engineering of the linker assembly, we construct the (G₄S)ₓ linker, where x indicates the degree of polymerization of the monomer sequence. The assembly is engineered like any other, and it falls into the scheme shown in FIG. 1. The specifics are shown in FIG. 19. Two synthetic DNA sequences are needed, SEQ. ID NO: 56 and SEQ ID NO: 57.

The first, the 5′-terminal cassette labeled as p0D11A in FIG. 19, is the sequence enabling addition of the linker sequence to other cassettes. It is flanked by a NcoI site, and thus with an upstream NdeI site, for cloning flexibility at the 5′ terminus, contains the NaeI site to create the blunt end ligation with the glycine codon at the 5′ terminus, the linker sequence, and finally the BamHI site within the GS codons. Plasmid p04 is prepared by digestion with NgoMIV, digestion with Mung Bean Nuclease, and finally re-ligation to destroy the internal NaeI site, creating plasmid p04A3. This altered plasmid, along with the insert, is digested with NcoI and BamHI and the appropriate fragments are ligated together. The resulting plasmid is labeled p0D11A. The open reading frame translation between the cleaved NaeI and the entire BamHI sites is G₄S.

SEQ ID NO: 57 is the sequence for the amplification cassette to create multimers of the G₄S linker. It is flanked by an NcoI site, again for cloning flexibility. It has the 5′ BclI site from the restriction pair, followed by the G₄S coding sequence that ends with the BamHI site. It is inserted into p04 by cutting both plasmid and insert with NcoI and BamHI and ligating the appropriate fragments together, as shown in FIG. 19. The resulting plasmid is labeled p0D11B.

Amplification cassette p0D11B is polymerized by the scheme shown in FIG. 3, left hand side, to create a dimer. In this instance the decision to follow the left hand side scheme results in larger fragments that are easier to isolate. Plasmid p0D11B is digested with NdeI and BclI and the large fragment is isolated. Separately, the same parent plasmid is digested with NdeI and BamHI, this time isolating the small fragment. The two isolated fragments are then ligated together, destroying the internal BclI and BamHI sites, but preserving the flanking ones. The resulting plasmid is labeled p0D11B2, the DNA insert is listed in SEQ ID NO: 58, and the ORF translation is listed in SEQ ID NO: 59. The sequence codes for the dimer (G₄S)₂. The process can be repeated with different starting cassettes to generate any (G₄S)ₓ linker. In this manner, (G₄S)₄ can be generated by digesting p0D12B with NdeI and BclI and saving the large fragment and ligating in the small fragment generated by digesting it with NdeI and BamHI.

The engineering of the G₄S assembly enables the construction of a GH multimer assembly with the (G₄S)₃ linker. The (G₄S)₃ 5′-terminal cassette for ligation to the GH sequences is generated following the general scheme shown in FIG. 4. Plasmid p0D11B2 is digested with NdeI and BclI, and the large fragment is isolated. The small fragment resulting from digestion of p0D11A with NdeI and BamHI is ligated in, creating plasmid p0D13A. The DNA and ORF sequences for the insert are listed in SEQ ID NO: 60 and SEQ ID NO: 61, respectively. The insert in p0D13A enables ligation of the (G₄S)₃ linker to the 3′ end of any sequence ending in a blunt end.

### Example 11

Engineering of the GH (G₄S)₃ assembly requires two new ends to the GH gene. The BclI 5′ restriction pair member is needed on the 5′ terminus of the amplification and 3′-terminal cassettes, and a blunt end immediately after the last codon of GH is needed on the 3′ terminus of the 5′-terminal and amplification cassettes for ligation of the (G₄S)₃ linker. There are many ways to get a blunt end at the 3′ terminus of GH. Disclosed here is the use of an NcoI site that is made blunt after digestion with Mung Bean Nuclease. In addition, it is convenient to introduce a stop codon flanked by the SalI restriction site at the 3′ terminus of the GH gene for construction of an insertion cassette, as shown in general in FIG. 5.

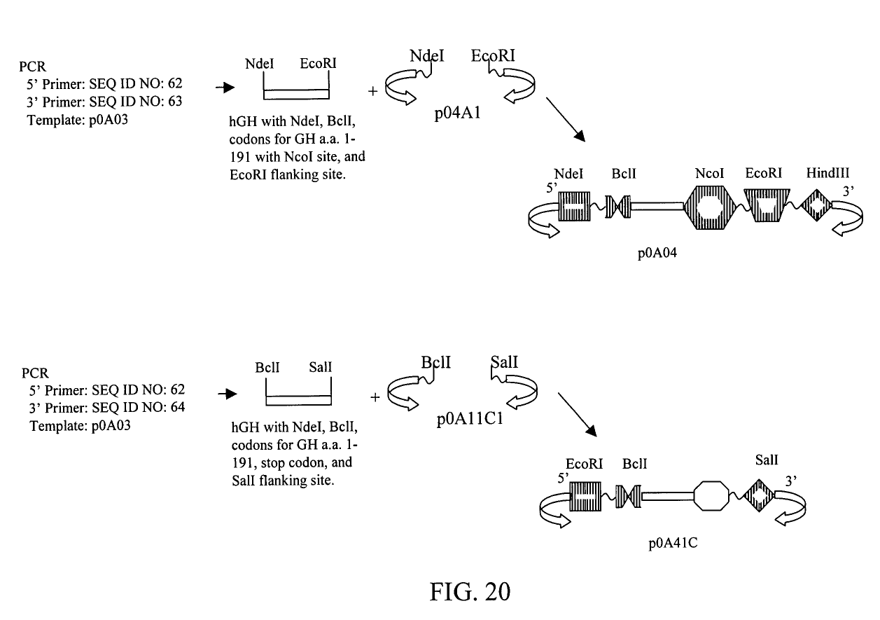

Three new primers are used to generate the new termini on two new GH inserts by PCR using P0A03 as template, as shown in FIG. 20. The 5′ primer is listed in SEQ ID NO: 62. It contains a flanking NdeI site, the BclI 5′ restriction pair member, and sequence complementary to the GH 5′ terminus. It is used for both PCR reactions. The 3′ primers are listed in SEQ ID NO: 63 and SEQ ID NO: 64. Both contain sequence complementary to the 3′ terminus of GH. The first codes for the NcoI site at the 3′ terminus for creation of a blunt end after the last GH base pair and a flanking EcoRI site, while the second introduces a stop codon followed by a SalI restriction site.

The PCR fragments are ligated into plasmid backbones as shown in FIG. 20. The PCR fragment resulting from use of the primers listed in SEQ ID NO: 62 and SEQ ID NO: 63 is digested with NdeI and EcoRI and ligated into similarly digested p04A1 to yield p0A04, while the fragment resulting from use of the primers listed in SEQ ID NO: 62 and SEQ ID NO: 64 is digested with BclI and SalI and ligated into similarly digested p0A11C1 to give p0A41C. The insert in p0A04 between the BclI and blunt ended NcoI sites has the DNA sequence listed in SEQ ID NO: 65 and corresponding ORF translation listed in SEQ ID NO: 66. Likewise, the insert in p0A41C, the 3′-terminal cassette, between the BclI and SalI sites has the DNA sequence listed in SEQ ID NO: 67 and ORF translation listed in SEQ ID NO: 68.

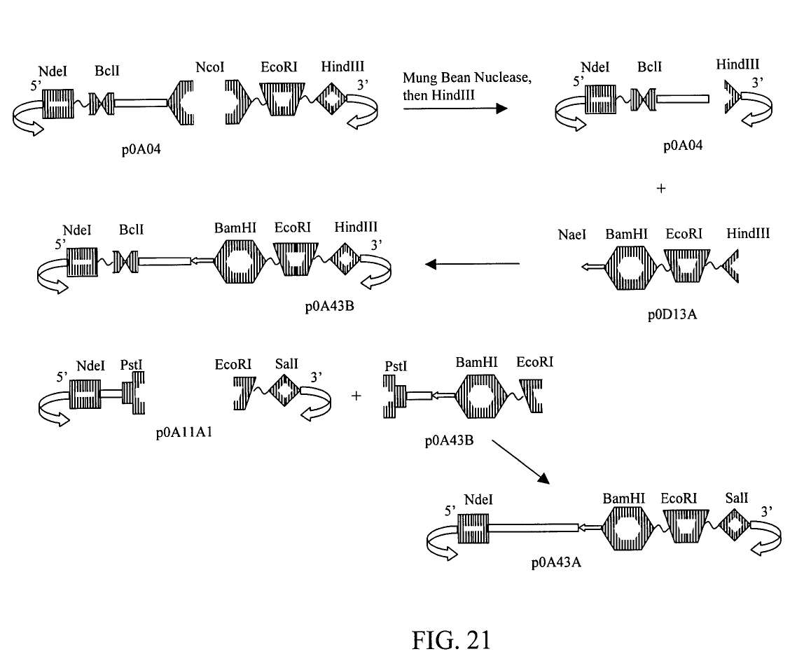

The amplification cassette is generated first by ligating the (G₄S)₃ linker from plasmid p0D13A with the insert in p0A04, as shown in FIG. 21. Plasmid p0D13A is digested with NaeI and HindIII, and the small fragment is isolated. It is ligated into p0A04 after digestion first with NcoI, then Mung Bean Nuclease, and finally HindIII to yield p0A43B. The resulting DNA sequence for the amplification cassette between the BclI and BamHI sites is listed in SEQ ID NO: 69, with corresponding ORF translation in SEQ ID NO: 70.

The direct start 5′-terminal cassette is generated by combining the 5′ elements from p0A11A1 with the 3′ elements from p0A43B, as shown in FIG. 21. The small fragment resulting from digesting p0A43B with PstI and EcoRI is isolated. It is ligated to the large fragment resulting from digestion of p0A11A1 with the same enzymes to yield p0A43A. The DNA sequence for the insert between NdeI and BamHI is listed in SEQ ID NO: 71, with corresponding ORF translation in SEQ ID NO: 72.

The polymerization of the amplification cassettes again follows the scheme in FIG. 3. The general formulas for the insert DNA and corresponding ORF translation for the Nmer amplification cassette are listed in SEQ ID NO: 73 and SEQ ID NO: 74.

The ligation of the cassettes for the GH (G₄S)₃ linker assembly to create a multimer expression cassette follows the previously described scheme shown in FIG. 7 and demonstrated in Example 4. The insertion cassette is first generated with the 5′- and 3′-terminal cassettes using EcoRI and SalI digestions. An amplification cassette insert is first isolated after digestion with BclI and EcoRI and then spliced into the insertion cassette after digestion using BamHI and EcoRI. The resultant construct is subsequently digested with BamHI and BclI and re-ligated. The resulting N+2 multimer expression cassette, where N is the degree of polymerization of the amplification cassette used, has DNA and corresponding ORF translation sequences listed in SEQ ID NO: 75 and SEQ ID NO: 76. Transfer of the insert into a suitable expression system yields multimeric GH with (G₄S)₃ linker.

### Example 12

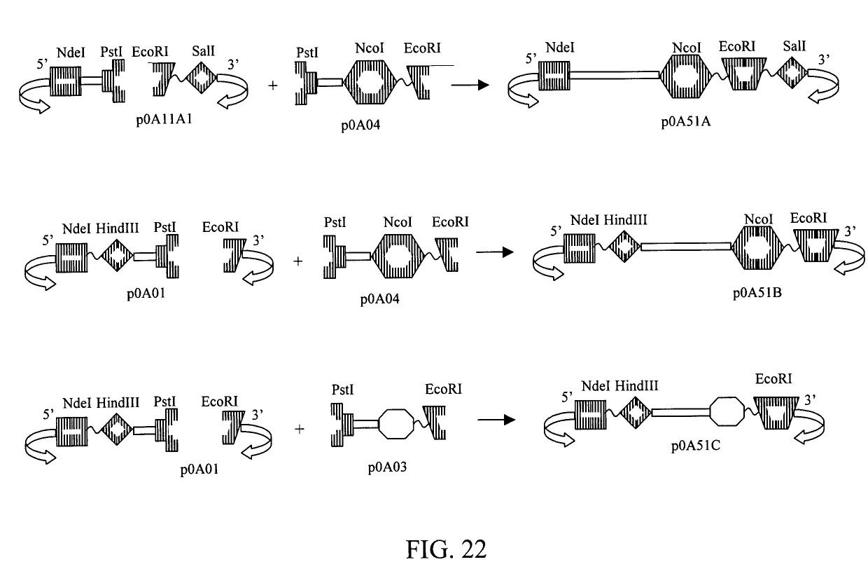

The last example is an alternative construction for a GH direct fusion assembly. It involves the use of an incompatible restriction pair that is blunt ended for ligation. Construction of this new assembly is done by ligating together fragments from earlier cassettes, since they already contain the needed elements. The construction scheme is shown in FIG. 22.

The 5′-terminal cassette is labeled p0A51A. It is generated by combining elements from p0A11A1 and p0A04. Plasmid p0A11A1 is digested with PstI and EcoRI and the open plasmid isolated. This is ligated with the insert isolated after digesting p0A04 with the same enzymes. The result, p0A51A, has DNA and corresponding ORF translation listed in SEQ ID NO: 77 and SEQ ID NO: 78.

The amplification and 3′-terminal cassettes are constructed in exactly the same manner as the 5′-terminal cassette, except for substituting which plasmids are digested. For the amplification cassette, plasmid p0A01 is ligated with the insert from p0A04. The insert DNA and corresponding ORF sequences are listed in SEQ ID NO: 79 and SEQ ID NO. 80. Likewise, for the 3′-terminal cassette, plasmid p0A01 is ligated with the insert from p0A03. Its insert DNA and corresponding ORF translation are listed in SEQ ID NO: 81 and SEQ ID NO: 82.

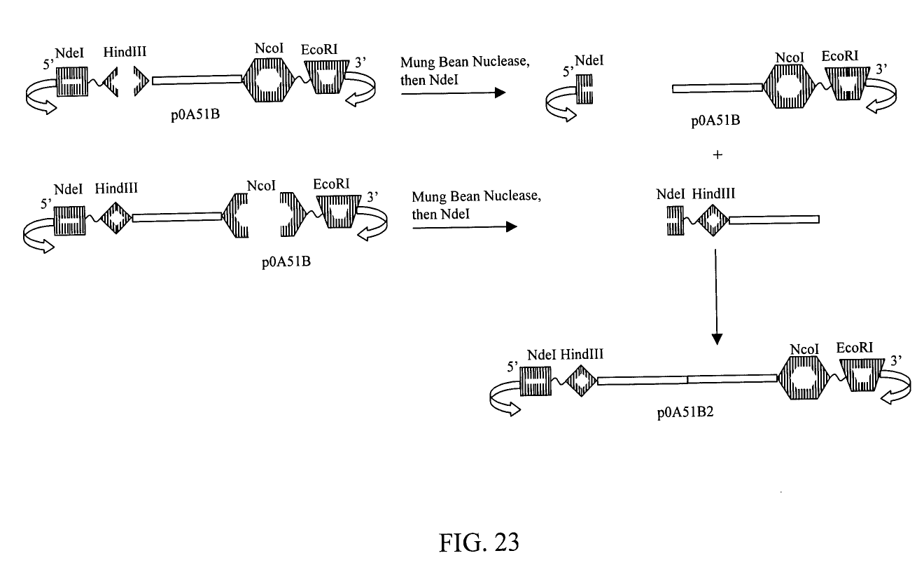

The polymerization of amplification cassettes still follows the scheme in FIG. 3. However, digestion at a restriction pair member now requires the additional blunt ending of its overhang. FIG. 23 shows the specifics for the current assembly. The digestions of the cassette are done sequentially so that the restriction pair is blunt ended, but the flanking restriction sites are left intact. The general formulas for the amplification cassettes are listed in SEQ ID NO: 83 and SEQ ID NO: 84.

The ligation of the multimer assembly cassettes is done sequentially as shown in FIG. 4. The digestion of any plasmid is performed as described above with blunt ending of the restriction pair member first. The general formulas for the resulting multimer expression cassette insert, using the Nmer amplification cassette, are listed in SEQ ID NO: 85 and SEQ ID NO: 86.

In practice, ligations of cassettes from this assembly involves more steps, but the technique's almost universal applicability may make it the method of choice in some instances. For the current case, the assembly given in Examples 1-4 is easier to manipulate.

Those skilled in the art will recognize many equivalents to the examples presented herein, using different monomers, linkers, restriction pairs, flanking restriction sites, 5′ specific sequences, 3′ specific sequences, and ligation strategies. For example, the methods are flexible as to the order of ligating 5′-terminal cassettes, 3′-terminal cassettes, and amplification cassettes, and in ligating amplification cassettes to one another to form higher order amplification cassettes. Combining elements of the following claims presented here and in the description, including the examples, is within the scope of the invention and are encompassed in the following claims.

All references cited herein, including the bibliography, are incorporated by reference in their entireties.

### References Cited

### U.S. PATENT DOCUMENTS

| | | |
|---|---|---|
| 5,084,390 | January 1992 Hallewell et al. | 435/188 |
| 5,876,969 | March 1999 Fleer et al. | 435/69.7 |
| 6,242,570 | June 2001 Sytkowski | 530/350 |

### OTHER PUBLICATIONS

Jorgensen, J. O. L., “Human Growth Hormone Replacement Therapy: Pharmacological and Clinical Aspects,” Endocrine Reviews, 12(3):189, (1991).

Chien, Y. W., “Human Insulin: Basic Sciences to Therapeutic Uses,” Drug Development and Industrial Pharmacy, 22(8):753-789 (1996).

Putney, S. D., and P. Burke, “Improving Protein Therapeutics with Sustained-release Formulations,” Nature Biotechnology, 16:153, (1998).

Johnson, O. L., et al., “A Month-long Effect from a Single Injection of Microencapsulated Human Growth Hormone,” Nature Medicine, 2(7):795, (1996).

Chen, S. A., et al., “Plasma and Lymph Pharmacokinetics of Recombinant Human Interleukin-2 and Polyethylene Glycol-modified Interleukin-2 in Pigs,” The Journal of Pharmacology and Experimental Therapeutics, 293(1):248, (2000).

Venkatachalam, M. A., and H. Rennke, “The Structural and Molecular Basis of Glomerular Filtration,” Circulation Research, 43(3):337-347, (1978).

Roberts, M. J., et al., “Chemistry for Peptide and Protein PEGylation,” Adv Drug Deliv Rev, 54(14):459-76, (2002).

Sharieff, K. A., et al., “Advances in Treatment of Chronic Hepatitis C: ‘Pegylated’ Interferons,” Cleve Clin J Med, 69(2):155-9, (2002).

Lewis, R. V., et al., “Expression and Purification of a Spider Silk Protein: A New Strategy for Producing Repetitive Proteins,” Protein Expression and Purification, 7:400-6, (1996).

Elmorjani, K., et al., “Synthetic Genes Specifying Periodic Polymers Modeled on the Repetitive Domain of Wheat Gliadins: Conception and Expression,” Biochemical and Biophysical Research Communication, 239:240-6, (1997).

Pennell, C. A., and P. Eldin, “In vitro Production of Recombinant Antibody Fragments in Pichia pastoris,” Res Immunol, 149(6):599-603, (1998).

Sambrook, et al., “Molecular Cloning: A Laboratory Manual,” Cold Spring Harbor Laboratory Press, NY, (2000).

Aggarwal, B. B. and Gutterman, J. U. Eds., “Human Cytokines: Handbook for Basic and Clinical Research,” Blackwell Scientific Publications, Boston, Mass., (1992).

What is claimed is:

1. A multimer assembly of DNA sequences comprising:
at least one amplification cassette, wherein said at least one amplification cassette comprises at least one monomer sequence whose polymerization is desired, further wherein said at least one amplification cassette comprises a 5′ restriction pair member at its 5′ terminus and a 3′ restriction pair member at its 3′ terminus; and at least one of the following:
at least one 3′-terminal cassette, wherein said 3′-terminal cassette comprises at least one 3′ specific sequence and a 5′ restriction pair member site that can be fused to a 3′ restriction pair member site of at least one of said at least one amplification cassette; or
at least one 5′-terminal cassette, wherein said 5′-terminal cassette comprises at least one 5′ specific sequence and a 3′ restriction pair member site that can be fused to a 5′ restriction pair member site of at least one of said at least one amplification cassette.

2. The multimer assembly of claim 1, wherein said at least one amplification cassette is at least two amplification cassettes.

3. The multimer assembly of claim 2, wherein said at least two amplification cassettes are fused at restriction pair member partners.

4. The multimer assembly of claim 1, wherein said multimer assembly comprises said at least one 5′-terminal cassette.

5. The multimer assembly of claim 1, wherein said multimer assembly comprises at least one 3′-terminal cassette.

6. The multimer assembly of claim 5, wherein said multimer assembly further comprises at least one 5′-terminal cassette.

7. The multimer assembly of claim 1, wherein said 5′ restriction pair member site and said 3′ restriction pair member site comprise:
ligation-compatible non-regenerable overhang restriction sites;
ligation-compatible non-regenerable blunt end restriction sites; or
incompatible overhang restriction sites that are converted to ligation-compatible non-regenerable blunt end restriction sites through the use of polymerases or nucleases.

8. The multimer assembly of claim 7, wherein said 5′ restriction pair member site and said 3′ restriction pair member site comprise ligation-compatible non-regenerable overhang restriction sites.

9. The multimer assembly of claim 7, wherein said 5′ restriction pair member site and said 3′ restriction pair member site comprise ligation-compatible non-regenerable blunt end restriction sites.

10. The multimer assembly of claim 7, wherein said 5′ restriction pair member site and said 3′ restriction pair member site comprise incompatible overhang restriction sites that are converted to ligation-compatible non-regenerable blunt end restriction sites through the use of polymerases or nucleases.

11. The multimer assembly of claim 4, wherein said 5′-terminal cassette further comprises at least a portion of said monomer sequence.

12. The multimer assembly of claim 5, wherein said 3′-terminal cassette further comprises at least a portion of said monomer sequence.

13. The multimer assembly of claim 6, wherein said 3′-terminal cassette and said 5′-terminal cassette each comprise at least a portion of said monomer sequence.

14. The multimer assembly of claim 1, wherein said multimer assembly further comprises at least one linker.

15. The multimer assembly of claim 14, wherein said at least one linker comprises at least one restriction pair member.

16. The multimer assembly of claim 1, wherein said monomer sequence encodes a peptide or protein of interest.

17. The multimer assembly of claim 16, wherein said peptide or protein of interest comprises at least a portion of a diagnostic protein.

18. The multimer assembly of claim 17, wherein said diagnostic protein is a cytokine, a hormone, a receptor, a receptor ligand, an enzyme, an inhibitor, a transcription factor, a translation factor, a DNA replication factor, an activator, a chaperone, or an antibody.

19. The multimer assembly of claim 16, wherein said peptide or protein of interest comprises at least a portion of a therapeutic protein.

20. The multimer assembly of claim 19, wherein said therapeutic protein is a cytokine, a growth factor, a hormone, a receptor, a receptor ligand, an enzyme, an inhibitor, a transcription factor, a translation factor, a DNA replication factor, an activator, a chaperonin, or an antibody.

21. The multimer assembly of claim 20, wherein said therapeutic protein is Interferon alpha., Interferon-beta., Interferon-gamma., Interleukin-1, Interleukin-2, Interleukin-3, Interleukin-4, Interleukin-5, Interleukin-6, Interleukin-7, Interleukin-8, Interleukin-9, Interleukin-10, Interleukin-11, Interleukin-12, Interleukin-13, Interleukin-14, Interleukin-15, Interleukin-16, Erythropoietin, Colony-Stimulating Factor-1, Granulocyte Colony-stimulating Factor, Granulocyte-Macrophage Colony-Stimulating Factor, Leukemia Inhibitory Factor, Tumor Necrosis Factor, Lymphotoxin, Platelet-Derived Growth Factor, Fibroblast Growth Factors, Vascular Endothelial Cell Growth Factor, Epidermal Growth Factor, Transforming Growth Factor-beta., Transforming Growth Factor-alpha., Thrombopoietin, Stem Cell Factor, Oncostatin M, Amphiregulin, Mullerian-Inhibiting Substance, B-Cell Growth Factor, Macrophage Migration Inhibiting Factor, Endostatin, or Angiostatin.

22. The multimer assembly of claim 1, wherein said 3′-restriction pair member encodes a stop codon that is destroyed upon ligation to said 5′-restriction pair member.

23. An amplification cassette comprising a 5′ segment of a monomer sequence and a 3′ segment of a monomer sequence that together comprise the sequence of a complete monomer, wherein said 5′ segment is positioned 3′ of said 3′ segment, further wherein 5′terminus of said 3′ segment is a 5′ restriction pair member and the 3′ terminus of said 5′ segment is a 3′ restriction pair member.

24. The multimer assembly of claim 1, wherein said multimer assembly comprises an amplification cassette that comprises a 5′ segment of a monomer sequence and a 3′ segment of a monomer sequence that together comprise the sequence of a complete monomer, wherein said 5′ segment is positioned 3′ of said 3′ segment, further wherein 5′terminus of said 3′ segment is a 5′ restriction pair member and the 3′ terminus of said 5′ segment is a 3′ restriction pair member.

25. The multimer assembly of claim 24, wherein said multimer assembly comprises:
at least one amplification cassette, wherein said at least one amplification cassette comprises at least one monomer sequence whose polymerization is desired, further wherein said at least one amplification cassette comprises a 5′ restriction pair member at its 5′ terminus and a 3′ restriction pair member at its 3′ terminus;
a 3′-terminal cassette, wherein said 3′-terminal cassette comprises said 3′ segment; and
a 5′-terminal cassette, wherein said 5′-terminal cassette comprises said 5′ segment.

26. The multimer assembly of claim 24, wherein said amplification cassette comprises a linker that is positioned between said 5′ segment and said 3′ segment of said monomer sequence.

27. The multimer assembly of claim 1, wherein said multimer assembly comprises a first cassette and a second cassette,
wherein when said first cassette comprises a 5′-terminal cassette, said second cassette comprises an amplification cassette or a multimer cassette constructed from a 3′-terminal cassette and an amplification cassette; and when said first cassette comprises a 3′-terminal cassette, said second cassette comprises an amplification cassette or a multimer cassette constructed from a 5′-terminal cassette and an amplification cassette.

28. A method of making a multimer cassette from a multimer assembly of claim 27, comprising:
a) digesting said first cassette at said 5′-restriction pair member or said 3′-restriction pair member and isolating a first fragment containing the insert sequence from said first cassette;
b) digesting said second cassette at said 5′ restriction pair member site and said 3′ restriction pair member site and isolating a second fragment containing the insert sequence from said second cassette;
c) ligating said first fragment with said second fragment to generate multimer cassette candidates; and
d) testing said multimer cassette candidates for correct ligation orientation, wherein a multimer cassette candidate with correct ligation orientation comprises a multimer cassette.

29. A multimer cassette made by the method of claim 28.

30. The method of claim 28 wherein said first cassette is a 3′-terminal cassette and said second cassette is an amplification cassette.

31. A multimer cassette made by the method of claim 30.

32. The method of claim 28 wherein said first cassette is a 5′-terminal cassette and said second cassette is an amplification cassette.

33. A multimer cassette made by the method of claim 32.

34. The method of claim 28 wherein said first cassette is a 3′-terminal cassette and said second cassette is a multimer cassette constructed from a 5′-terminal cassette and an amplification cassette.

35. A multimer cassette made by the method of claim 34.

36. The method of claim 28 wherein said first cassette is a 5′-terminal cassette and said second cassette is a multimer cassette constructed from a 3′-terminal cassette and an amplification cassette.

37. A multimer cassette made by the method of claim 36.

38. The multimer assembly of claim 1, wherein at least one of said cassettes comprises one or more flanking restriction sites.

39. The multimer assembly of claim 4, wherein at least one of said cassettes comprises one or more flanking restriction sites.

40. The multimer assembly of claim 5, wherein at least one of said cassettes comprises one or more flanking restriction sites.

41. The multimer assembly of claim 6, wherein at least one of said cassettes comprises one or more flanking restriction sites.

42. The multimer assembly of claim 6, wherein said 5′-terminal cassette and said 3′-terminal cassette each contain the same insertion restriction site.

43. A method of making a multimer cassette from two cassettes from a multimer assembly of claim 27, wherein each of said two cassettes comprises one or more flanking restriction sites, comprising:
a) providing a first cassette with a first flanking restriction site at one end, either 5′ or 3′, of its insert sequence;
b) providing a second cassette with a second flanking restriction site that is, or is made, ligation compatible with said first flanking restriction site and is on the same side, either 5′ or 3′, of its insert sequence as the first flanking restriction site is relative to said first cassette's insert sequence;
c) digesting said first cassette at its restriction pair member and said first flanking site and isolating the first fragment containing the insert sequence;
d) digesting said second cassette at its restriction pair member partner to said first cassette's restriction pair member and at said second flanking site and isolating the second fragment containing the insert sequence; and
e) ligating said first fragment with said second fragment to generate a multimer cassette.

44. A multimer cassette made by the method of claim 43.

45. The method of claim 43 wherein said first cassette is a 3′-terminal cassette and said second cassette is an amplification cassette.

46. The method of claim 43, wherein said first cassette is a 5′-terminal cassette and said second cassette is an amplification cassette.

47. The method of claim 43 wherein said first cassette is a 3′-terminal cassette and said second cassette is a multimer cassette constructed from a 5′-terminal cassette and an amplification cassette.

48. The method of claim 43 wherein said first cassette is a 5′-terminal cassette and said second cassette is a multimer cassette constructed from a 3′-terminal cassette and an amplification cassette.

49. A method of making an insertion cassette from the multimer assembly of claim 42 comprising:
a) providing said 5′-terminal cassette having a first flanking restriction site, distinct from said insertion restriction site, that is outside of the sequence including the insert sequence and insertion restriction site of said 5′-terminal cassette;
b) providing a 3′-terminal cassette having a second flanking restriction site, distinct from said insertion restriction site, that is outside of the sequence including the insert sequence and insertion restriction site of said 3′-terminal cassette and is, or is made, ligation compatible with said first flanking site and is on the same side, either 5′ or 3′, of its insert sequence as the first flanking restriction site is relative to said 5′-terminal cassette's insert sequence;
c) digesting said 5′-terminal cassette at its insertion restriction site and said first flanking site and isolating the first fragment containing the insert sequence;
d) digesting said 3′-terminal cassette at its insertion restriction site and said second flanking site and isolating the second fragment containing the insert sequence; and
e) ligating said first fragment with said second fragment to generate an insertion cassette.

50. An insertion cassette made by the method of claim 49.

51. A method of making a multimer cassette from a multimer assembly that comprises an amplification cassette and the insertion cassette of claim 50, comprising:
a) digesting said insertion cassette at both its restriction pair member sites and isolating a first fragment containing an insertion cassette insert sequence;
b) digesting said amplification cassette at both its said restriction pair member sites and isolating a second fragment containing an amplification cassette insert sequence; and
c) ligating said first fragment with said second fragment to generate multimer cassette candidates;
d) testing said multimer cassette candidates for correct ligation orientation, wherein said a multimer cassette candidate with correct ligation orientation comprises a multimer cassette.

52. A multimer cassette made by the method of claim 51.

53. A method of making a multimer cassette from a multimer assembly that comprises an amplification cassette and the insertion cassette of claim 50, comprising:
a) providing said amplification cassette comprising a flanking restriction site that is, or is made, ligation compatible to said insertion restriction site of said insertion cassette;
b) digesting said amplification cassette at said flanking restriction site and its restriction pair member on the opposite side, either 5′ or 3′, of the insert sequence and isolating the first fragment containing the insert sequence;
c) digesting said insertion cassette at said insertion restriction site and the restriction pair member partner to said digested amplification cassette's restriction pair member and isolating the second fragment containing the insert sequence;
d) ligating said first fragment with said second fragment to generate a multimer cassette precursor; and
e) digesting said multimer cassette precursor at both restriction pair members, isolating the fragment containing the insert sequence, and ligating it with itself to generate a multimer cassette.

54. A multimer cassette made by the method of claim 53.

55. A multimer assembly according to claim 1, wherein said monomer sequence is the hGH coding sequence, SEQ ID NO: 1.

56. The multimer assembly of claim 25, wherein said monomer sequence is the hGH coding sequence, SEQ ID NO: 1.

57. The multimer assembly of claim 56, wherein a restriction pair is utilized at the coding sequence of amino acids 187 and 188, glycine and serine, of monomeric hGH.

58. The multimer assembly of claim 57, wherein insert sequences lack linkers.

59. The multimer assembly of claim 58, wherein said 5′-terminal cassette is listed in SEQ ID NO: 15.

60. The multimer assembly of claim 58, wherein said 5′-terminal cassette is listed in SEQ ID NO: 17.

61. The multimer assembly of claim 58, wherein the general formula for said amplification cassette is listed, in SEQ ID NO: 28.

62. The multimer assembly of claim 58, wherein said 3′-terminal cassette is listed in SEQ ID NO: 22.

63. The multimer assembly of claim 58, wherein a 3′-terminal cassette is listed in SEQ ID NO: 24.

64. The multimer assembly of claim 58, wherein the insertion cassette is listed in SEQ ID NO: 30.

65. The multimer assembly of claim 58, wherein the general formula for the multimer expression cassettes is listed in SEQ ID NO: 31.

66. A multimer expression cassette made from the multimer assembly of claim 58.

67. A polymeric protein expressed from the multimer expression cassette of claim 66 as described by SEQ ID NO: 32.

68. The multimer assembly of claim 57, wherein at least one insert sequence comprises at least one linker.

69. The multimer assembly according to claim 68, wherein said linker is the single amino acid glycine.

70. The multimer assembly of claim 69, wherein said 5′-terminal cassette is listed in SEQ ID NO: 15.

71. The multimer assembly of claim 69, wherein said 5′-terminal cassette is listed in SEQ ID NO: 17.

72. The multimer assembly of claim 69, wherein the general formula for the amplification cassettes is listed in SEQ ID NO: 36.

73. The multimer assembly of claim 69, wherein a 3′-terminal cassette is listed in SEQ ID NO: 22.

74. The multimer assembly of claim 69, wherein said 3′-terminal cassette is listed in SEQ ID NO: 24.

75. The multimer assembly of claim 69, wherein the insertion cassette is listed in SEQ ID NO: 30.

76. The multimer assembly of claim 69, wherein the general formula for the amplification cassettes is listed in SEQ ID NO: 38.

77. A multimer expression cassette made from the multimer assembly of claim 69.

78. A polymeric protein expressed from the multimer expression cassette of claim 77 as described by SEQ ID NO: 39.

79. The multimer assembly of claim 22, wherein at least one said insert sequence comprises a linker coding for the peptide sequence A-Ser-Trp-B, where A and B are arbitrary peptide sequences, the 3′-restriction pair member is RcaI, T\hat{ }CATGA, and the 5′-restriction pair member is NcoI, C\hat{ }CATGG.

80. A multimer assembly according to claim 79, wherein said monomer sequence encodes hGH or a portion thereof.

81. The multimer assembly of claim 80, wherein said linker codes for the peptide Ser-Trp-Gly-Gly-Gly-Gly-Ser.

82. The multimer assembly of claim 80, wherein a 5′-terminal cassette is listed in SEQ ID NO: 41.

83. The multimer assembly of claim 80, wherein the general formula for the amplification cassettes is listed in SEQ ID NO: 48.

84. The multimer assembly of claim 80, wherein a 3′-terminal cassette is listed in SEQ ID NO: 46.

85. The multimer assembly of claim 80, wherein the general formula for multimer expression cassettes is listed in SEQ ID NO: 52.

86. The multimer assembly of claim 80, wherein the general formula for multimer expression cassettes is listed in SEQ ID NO: 54.

87. A multimer expression cassette made from the multimer assembly of claim 80.

88. A polymeric protein expressed from the multimer expression cassette of claim 86 as described by SEQ ID NO: 53.

89. A polymeric protein expressed from the multimer expression cassette of claim 86 as described by SEQ ID NO: 55.

90. A multimer assembly according to claim 1 comprising at least one linker sequence adjacent to at least one monomer sequence of at least one amplification cassette.

91. The multimer assembly according to claim 5, further comprising at least one linker sequence adjacent to at least one monomer sequence of at least one amplification cassette.

92. The multimer assembly according to claim 91, wherein said linker comprises at least one restriction site compatible with a restriction site of said 3′-terminal cassette.

93. The multimer assembly according to claim 4, further comprising at least one linker sequence adjacent to at least one monomer sequence of at least one amplification cassette.

94. The multimer assembly according to claim 93, wherein said linker comprises at least one restriction site compatible with a restriction site of said 5′-terminal cassette.

95. The multimer assembly according to claim 94, wherein said linker codes for the peptide sequence (GZGS)ₓ, where Z is an arbitrary sequence of arbitrary length and x indicates the degree of polymerization of the peptide monomer sequence.

96. The multimer assembly according to claim 95, wherein Z is GG.

97. The multimer assembly of claim 96, wherein a 5′-terminal cassette is listed in SEQ ID NO: 56

98. The multimer assembly of claim 96, wherein an amplification cassette is listed in SEQ ID NO: 57

99. The multimer assembly of claim 96, wherein an amplification cassette is listed in SEQ ID NO: 58

100. The multimer assembly of claim 96, wherein a multimer cassette is listed in SEQ ID NO: 60.

101. The multimer assembly according to claim 15, wherein said monomer sequence encodes hGH or a portion thereof.

102. The multimer assembly according to claim 101, wherein said linker codes for the peptide (G₄S)₃.

103. The multimer assembly according to claim 102, wherein a 5′-terminal cassette is listed in SEQ ID NO: 71

104. The multimer assembly according to claim 102, wherein the general formula for amplification cassettes is listed in SEQ ID NO: 73,

105. The multimer assembly according to claim 102, wherein a 3′-terminal cassette is listed in SEQ ID NO: 67.

106. The multimer assembly according to claim 102, wherein a general formula for multimer expression cassettes is listed in SEQ ID NO: 75.

107. A multimer expression cassette made from the multimer assembly of claim 106.

108. A polymeric protein expressed from the multimer expression cassette of claim 107 as described by SEQ ID NO: 76.

109. The multimer assembly according to claim 10, wherein said monomer sequence encodes hGH or a portion thereof.

110. The multimer assembly of claim 109, wherein a 5′-terminal cassette is listed in SEQ ID NO: 77.

111. The multimer assembly of claim 110, wherein a general formula for said amplification cassettes is listed in SEQ ID NO: 83.

112. The multimer assembly of claim 110, wherein a 3′-terminal cassette is listed in SEQ ID NO: 81.

113. The multimer assembly of claim 110, wherein a general formula for the multimer expression cassettes is listed in SEQ ID NO: 85.

114. A multimer expression cassette made from the multimer assembly of claim 113.

115. A polymeric protein expressed from the multimer expression cassette of claim 114 as described by SEQ ID NO: 86.

116. A multimer cassette made by the method of claim 45.

117. A multimer cassette made by the method of claim 46.

118. A multimer cassette made by the method of claim 47.

119. A multimer cassette made by the method of claim 48.

120. A vector comprising a multimer cassette made from a multimer assembly of any of claims 1-27, 38-42, 55-65, 68-76, 79-86,90-106, or 109-113.

121. The vector of claim 120, wherein said vector is an expression vector, wherein said expression vector is designed for in vitro or in vivo expression.

122. A cell containing a vector according to claim 121.

123. A polymeric protein expressed from a vector of claim 122.

124. A method of making an amplification cassette, comprising:
a) providing at least two amplification cassettes of claim 23; and
b) joining said at least two amplification cassettes by ligating said 3′ restriction member of at least one of said at least two amplification cassettes to said 5′ restriction member of at least one other of said at least two amplification cassettes to generate a multimer cassette.

125. An amplification cassette comprising at least one linker, wherein said at least one linker comprises at least one restriction pair member member.

126. A method of making an amplification cassette, comprising:
a) providing at least two amplification cassettes of claim 125; wherein each of said at least two amplification cassettes comprises: a first restriction pair partner on one end of said monomer sequence; and a linker at the other end of said monomer sequence that comprises a second restriction pair partner; and
b) joining said at least two amplification cassettes by ligating said first restriction pair partner of at least one of said at least two amplification cassettes to said second restriction pair partner of at least one other of said at least two amplification cassettes to generate a multimer cassette.

127. An amplification cassette comprising 5′ and 3′ restriction pair member sites that are incompatible overhang restriction sites that are converted to ligation-compatible nonregenerable blunt end restriction sites through the use of polymerases or nucleases.

128. A method of making an amplification cassette, comprising:
a) providing at least two amplification cassettes of claim 127; and
b) joining said at least two amplification cassettes by ligating said 3′ restriction member of at least one of said at least two amplification cassettes to said 5′ restriction member of at least one other of said at least two amplification cassettes to generate a multimer cassette.

129. A vector comprising a multimer assembly cassette of any of claims 23, 29, 31, 33, 35, 37, 44, 50, 52, 54, 66, 77, 87, 107, 116-119, 125, or 127.

130. The vector of claim 129, wherein said vector is an expression vector, wherein said expression vector is designed for in vitro or in vivo expression.

131. A cell containing a vector according to claim 130.

132. A polymeric protein expressed from the vector of claim 131.
